(19) United States
(12) Patent Application Publication
(10) Pub. No.: US 2005/0202032 A1
(43) Pub. Date: Sep. 15, 2005

(54) Use of neglected target tissue antigens in modulation of immune responses

(75) Inventors: Daniel Kaufman, Santa Monica, CA (US); Jide Tian, Los Angeles, CA (US); Angelica Olcott, Los Angeles, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/754,473

(22) Filed: Jan. 9, 2004

Related U.S. Application Data
(62) Division of application No. 09/310,707, filed on May 7, 1999, now abandoned.
(60) Provisional application No. 60/084,636, filed on May 7, 1998.

(51) Int. Cl. A61K 39/00

(52) U.S. Cl. 424/185.1

(57) ABSTRACT

Disclosed are methods for identifying antigens, termed neglected target tissue antigens, that do not become involved as targets of an abnormal immune response (such as allergy or autoimmunity or more generally inflammation); also disclosed are methods of using NTTAs for inducing regulatory responses and thereby abating abnormal inflammatory immune responses.

### U.S. GOVERNMENT RIGHTS

This invention was made with Government support under Grant No. DK 48455 awarded by the National Institutes of Health. The U.S. Government has certain rights in this invention.

### FIELD OF THE INVENTION

This invention relates to the use of certain non-target antigens in modulating immune responses, and more specifically in abating undesirable immune responses, such as autoimmunity, especially during late stages in an autoimmunity cascade.

### BACKGROUND OF THE INVENTION

In experimentally induced organ-specific autoimmune disease models, in which the initiating antigen is defined, it is possible to circumvent the development of autoimmunity by deleting or inactivating T cells that are reactive to the initiating antigen (Sharma, S. D., Nag, B., Su, X. M. et al. (1991) Proc. Natl. Acad. Sci. U.S.A. 88, 11465-11469; Critchfield, J. M., Rackie, M. K., Zuniga-Pflucker, J. C. et al. (1994) Science 263, 1139-1143). However, an initiating target antigen has not been identified in several human T-cell mediated autoimmune diseases such as multiple sclerosis (MS), insulin-dependent diabetes mellitus (IDDM) or rheumatoid arthritis (RA), precluding attempts to circumvent the initiation of self-reactivity by specific T-cell deletion. Current therapeutic strategies accordingly focus on inhibiting active autoimmune responses in symptomatic, or presymptomatic, individuals who are at high risk of developing disease (for example, prediabetic individuals with circulating islet autoantibodies).

Pharmacological immunosuppression, anti-inflammatory cytokines and antibodies against T-cell surface molecules can inhibit pathogenic autoimmune responses in animal models; however, these approaches also broadly interfere with immune system function. Interference with effector T-cell receptors (TCRs), the major histocompatibility complex (MHC) or their peptide ligands, or induction of apoptosis through MHC-peptide conjugates, targets the autoreactive T-cell population more selectively and has shown efficacy in animal models (Sharma, supra; Zamvil S. et al., 1990, Annu. Rev. Immunol. 8:579; Gaur A. et al., 1994, Adv. Immunol. 56:219). However, such strategies are difficult to apply to the genetically diverse human patient population whose autoreactive T-cell repertoire may be changing and evolving with disease progression.

Studies of animal models of organ-specific autoimmune diseases suggest that a cascade of autoreactive Th1-type inflammatory responses mediates the disease process (4-8). Although the initial autoimmune response appears limited in its recognition of self-antigens, it subsequently expands to react against additional target tissue antigens (2, 5-9). Owing to this spreading of autoimmunity, symptomatic and autoantibody-positive presymptomatic individuals are likely to have a diverse autoreactive T-cell repertoire which makes it more difficult to delete or inactivate T-cells selectively.

Based on the antagonistic functions of different T-cell subsets, various researchers have proposed that autoantigen administration in modes that induce regulatory responses (such Th2, Th3, Tr1 or other anti-inflammatory cells) (9-12) could be used to down regulate pathogenic autoimmune responses and inhibit disease progression (13-16). This approach would not broadly interfere with immune system function, nor would it require information on the initiating target antigen or the specificity of the effector T cells, since the autoantigen-induced regulatory responses would, upon re-encountering their cognate antigen in a target organ, release anti-inflammatory cytokines that locally suppress effector T cells, regardless of their specificity, in a process termed ‘bystander suppression’ (Weiner, H. L., 1997, Immunol. Today, 18:335).

The ability of autoantigen-based immunotherapies to induce antigen-specific suppressor T cells which establish ‘regulatory tolerance’ and inhibit autoimmune-mediated tissue damage has been amply demonstrated in animal models. Indeed, many of the classic tolerization protocols for the treatment of autoimmune disease (such as autoantigen administration by intraperitoneal, oral or nasal routes) are now known to involve the induction of regulatory responses. (Weiner, H. L. (1997) Immunol. Today 18, 335-343; Holt, P. G. and McMenamin, C. (1989) Clin. Exp. Allergy 19, 255-262; Forsthuber, T., Yip, H. C. and Lehmann, P. V. (1966) Science 271, 1728-1730). These studies, as well as other studies that manipulated cytokines or accessory molecules. (Powrie, E. and Coffman, R. L. (1993) Immunol. Today 14,270-274; Lenschow, D. J., Herold, K. C. Rhee, L. et al. (1966) Immunity 5,285-293; Shaw, M. K., Lorens, J. B., Dhawan, A. et al. (1977) J. Exp. Med. 185, 1711-1714; Rapoport, M. J., Jaramillo, A., Zipris, D. et al. (1993) J. Exp. Med. 178, 87-99) demonstrated that the induction of regulatory responses could be a potent modulator of disease outcome. Although autoreactive pro-inflammatory T cell responses can persist long after treatment (e.g. refs (16-18)), the treated animals often remain disease-free, indicating that the induced regulatory responses can establish long-term functional tolerance.

Despite the success of antigen-based immunotherapies that were based on the immune deviation paradigm, the precise mode of action on antigen-based immuno therapeutics is highly debated. In different animal models, the induction of cells that secrete interleukin 4 (IL-4)(Th2), transforming growth factor β (TGF-β) (Th3), or IL-10 (T regulatory 1; Tr1), as well as suppressive CD8+ cells, has been associated with the inhibition of disease progression. Although antigen administration has been shown convincingly to induce cells that mediate bystander suppression in a target tissue, in vitro-differentiated T-cell clones (particularly Th2-type clones) often fail to mitigate target tissue damage caused by pathogenic Th1 cells in adoptive transfer experiments.

The nonspecific nature of bystander suppression (see, e.g., Int'l Pat. Applns WO95/27500 and WO93/16724), is attractive in that the administration of any target tissue antigen can elicit regulatory responses which will inhibit inflammation in the target tissue. Indeed, the administration of a variety of different β cell autoantigens (βCAAs) including GAD, HSP and insulin-B chain can be highly protective when given (intraperitoneally in incomplete Freund's adjuvant) early in the NOD mouse disease process (1). However, experiments by the present inventors and their co-workers showed that when βCAAs are administered late in the disease process, they differ greatly in their ability to prime anti-inflammatory responses and to prevent IDDM, as well as in their ability to protect transplanted syngeneic islets in diabetic NOD mice (2, 3) and FIG. 1.

Other researchers have also reported an inability to induce tolerance when the tolerizing antigen was administered orally after disease induction (Nagler-Anderson, 1986, PNAS, 83:7443; Bitar, D. M. et al. Cell. Immunol., 1988, 112:364), although these results cannot be easily attributed to a late stage of autoimmunity because there are several other variables that could have interfered.

As individuals who are identified as being at risk for insulin dependent diabetes mellitus (IDDM) by autoantibody screening are likely to be in advanced stages of the autoimmune cascade (and the same likelihood pertains to individuals who have autoreactivities associated with other autoimmune diseases), there is still a need to develop antigen-based immunotherapies that remain effective later in the course of disease progression, and specifically after autoimmunity has spread.

There have been some fears that administration of autoantigenic substances might, instead of inducing tolerance, boost pro-inflammatory autoimmune responses. To favor the induction of regulatory responses, researchers have used adjuvants and cytokines and routes of administration (e.g. oral route) that tend to promote anti-inflammatory responses. In many studies, the administration of autoantigens selectively engaged anti-inflammatory responses without boosting established proinflammatory responses or creating new pathologies. (Steinman, L. (1996) Proc. Natl. Acad. Sci. U.S.A. 93, 2253-2256; Tian, J., Clare-Salzler, M., Herschenfeld, A. et al. (1996) Nat. Med. 2, 1348-1353; Muir, A., Peck, A., Clare-Salzler, M. et al. (1995) J. Clin. Invest. 95, 628-634; Elias, D., Meilin, A., Ablamunits, V. et al. (1997) Diabetes 46, 758-764). Nevertheless, there are risks associated with any autoantigen administration.

Certain lines of research have indicated that administered autoantigens can modulate immune responses in ways that were not intended, even if such modulation is not shown to be harmful.

These results underscore the possibility that antigen-based immunotherapies might elicit immune responses in unexpected ways in the genetically diverse human population, in which, additionally, each individual has a unique immunological history of environmental exposures. However, autoantigens have been fed to over a thousand individuals with different types of autoimmune disease without reported toxicity or exacerbation of disease linked to treatment; the sole exception being a study that found mixed trends depending on the antigen preparation.

There have also been some questions about efficacy of administering autoantigens.

Accordingly, there is a need to develop effective antigen-based immunotherapies that are accompanied by fewer concerns about safety and efficacy.

### OBJECTS OF THE INVENTION

It is an object of the present invention to provide an effective antigen-based immunotherapy (both to develop immunotherapeutic agents and a method for their use) for immune system disorders including, without limitation, allergies and autoimmune diseases.

A more particular object is to provide an effective antigen-based immunotherapy for autoimmune diseases.

A still more particular object is to provide during a late stage of the autoimmunity cascade an effective antigen-based immunotherapy for a mammal (animal or human) suffering from an autoimmune disease.

Yet another object is to provide a safe antigen-based immunotherapy for a person or other mammal suffering from an immune system disorder.

Another object of the invention is to provide an effective antigen-based immunotherapy for immune system disorders even when target determinant-based immunotherapies have failed.

Another object of the invention is to provide an effective antigen-based immunotherapy for immune system disorders even late in the cascade of abnormal immune events.

Yet another object of the invention is to prime towards a desirable (regulatory) response T cells previously uncommitted to either regulation or enhancement of an (undesirable) immune response.

Further objects of the invention include taking advantage of previously untapped T cell populations of a host afflicted with an immune disorder and recruiting them towards regulation of abnormal immune responses.

### SUMMARY OF THE INVENTION

The present inventors noted experimental results which showed that the ability to mount vigorous Th2 responses late in the cascade of autoimmunity does not diminish vis-a-vis non-target antigens, but only diminishes towards autoantigens.

The present invention is based on the discovery that neglected target tissue antigens (NTTA), which are not involved (and do not become involved) in an autoimmune response even during late stages of the autoimmunity cascade (when autoimmunity has spread) can be highly effective immunotherapeutic agents. NTTAs can be used to induce regulatory immune responses which abate one or more autoimmune responses associated with an autoimmune disease.

The present inventors' data show inter alia peptides encompassing neglected determinants of antigens expressed in the target organ or tissue (i.e. determinants that do not become involved in the cascade of autoimmune responses whether they form part of an autoantigen or not) are capable of recognition by substantial populations of uncommitted T cells which can be primed, or biased, towards regulatory responses, and can provide an effective treatment. In particular, NTTA can be effective in regulating undesirable immune responses even when target determinants used as tolerizing agents have failed to induce an effective regulatory T cell response.

Use of NTTAs as tolerizing agents is anticipated to be safer than use of target determinants.

This discovery has wider applicability than autoimmune diseases because it involves a direct modulation of immune responses, and is independent of the ultimate cause of inflammatory immune responses. Thus, the teachings of this invention include not only methods for treating autoimmune disease by tolerizing administration to afflicted persons (or animals) of NTTA but also methods for treating other abnormal inflammatory immune responses by administration of NTTAs derived from the organ or tissue afflicted with the abnormal immune response.

Another aspect of the invention is directed to methods for identifying antigen-based immunotherapeutic agents suitable for use in abating undesirable immune responses such as autoimmune responses or allergies or more generally inflammation. This involves identifying whole antigens or segments of antigens expressed in the target tissue (not necessarily tissue-specific) that are not recognized by activated T cells or antibodies of a host afflicted with an abnormal immune response.

The methods of the invention are thus applicable to regulation of abnormal immune responses in general.

### BRIEF DESCRIPTION OF THE DRAWINGS

FIG. 1 is a graph of percent recurrence of hyperglycemia (diabetes) in diabetes NOD mice into which islets were transplanted. The mice had been treated (injected intraperitoneally) with β-galactosidase (black triangles; positive control); GAD (open circles); insulin B-chain (open squares); or HSP peptide 277 (heat shock protein peptide) (open diamonds); all in incomplete Freund's adjuvant.

FIG. 2 is a graph depicting attenuation of inducible Th2 immunity to β-cell autoantibody in NOD mice. The graph is plotted as mean number of IL-4 secreting spot forming colonies (SFC) of splenic Tcell/10⁶ splenic Tcells v. treatment age when the mice had been treated with insulin B-chain (open triangles); heat shock protein peptide (black triangles); GAD (black circles); the irrelevant antigen β-galactosidase (open squares); and HEL (hen egg-white lysozyme) (open circles).

FIG. 3 is a bar graph depicting Th2 spreading decline (reducing ability of elicited Th2 cells to regulate immune responses directed at other antigens) with disease progression, namely the mean number of IL-4 secreting SFC/10⁶ splenic Tcells for mice treated at different ages (neonatal; 6 weeks; 18 weeks) with GAD/IFA; HSP/IFA or insulin/IFA. The bars depict response to stimulation with GAD (black), HSP (stripes) or insulin when the mice had been treated with GAD, HSP or insulin.

FIG. 4 is a plot of proliferative T cell response (stimulation index) against neglected target tissue antigen (NTTA) concentration for various NTTAs (#2 black circles; #4 open squares; #6 open triangles; and #7 black triangles) showing that, when used as the immunizant, NTTAs are indeed immunogenic.

FIG. 5 is a graph depicting adoptive transfer of regulatory responses as measured by percent diabetes (hyperglycemia) incidence versus mouse age (weeks) when splenic T cells from NTTA- or control-treated mice were co-transferred with T cells from diabetic NOD mice into irradiated young NOD mice. Mouse serum albumin peptide 560-574 was used as a control (open circles).

FIG. 6 is a similar plot depicting percent diabetes (hyperglycemia) incidence versus mouse age when mice were treated with a control MSA peptide (black squares; top plot); a beta cell autoantigen target determinant (heat shock protein peptide 277; insulin B-chain, GAD peptide 35, GAD peptide 34; middle plot cluster) or a NTTA peptide (#2, 4, 6 or 7; lower plot cluster).

FIG. 7 is a plot of the proportion of mice remaining diabetes-free in a group treated with NTTA, or beta cell antibodies, or with MSA as a control (based on the same data as FIG. 6).

FIG. 8 is a plot showing that NTTAs inhibit IDDM when target GAD determinants (autoantigens) do not. Percent diabetes incidence is plotted against mouse age for groups of mice treated with MSA (positive control), GAD target determinants, GAD peptide 35, GAD peptide 34, GAD peptide 32 and GAD NTTA's: GAD peptide 18, GAD peptide 27.

FIG. 9 is a plot of the same data that gave rise to FIG. 8 but plotted as proportion of diabetes free mice against age for NTTA-treated (“neglected”) and target determinant-treated (“targeted”) or control-treated (MSA) mice.

FIG. 10 is a bar graph of spot forming colonies of splenic cells from mice neonatally injected with the control antigen β-galactosidase or the target antigens GAD or HSP or insulin B-chain and stimulated with GAD, HSP, or insulin B-chain. Top graph—FIG. 10A: IFNγ-secreting cells (Th1 responses); Bottom graph—FIG. 10B: IL-4 secreting cells (Th2 responses).

### DETAILED DESCRIPTION OF THE INVENTION

All literature and other references cited herein are incorporated by reference in their entirety.

Definitions

“Neglected Target Tissue Antigens” or “NTTA” are antigens (whole antigens or peptidic segments thereof) of an animal or human suffering from, or susceptible to (at risk for), an autoimmune disease. NTTA are characterized by their nonparticipation in an abnormal autoimmune response (especially a spontaneous autoimmune response) associated with an autoimmune disease. Thus, although NTTA are antigens in that they are capable of being recognized by the immune system, they do not become involved in autoimmunity (even in later stages of an autoimmune disease or model thereof wherein autoimmunity and specifically Th1 autoimmunity has spread to other antigens of the target organ or tissue, a phenomenon termed “determinant spreading”). NTTA are “thus” neglected in that they are ignored by autoimmune events. NTTA include not only whole neglected antigens that are expressed (not necessarily specifically expressed) in an organ or tissue that is the target of an autoimmune response but also peptidic fragments or portions of autoantigens that are ignored by the autoimmune response. Certain NTTAs may be “cryptic determinants” as defined below (but not all cryptic determinants are NTTAs). In a specific embodiment in which the autoimmune disease IDDM is involved, non-limiting examples of NTTAs include portions of the beta cell glutamic acid decarboxylase (“GAD”) (such as GAD peptide 18, or GAD peptide 27), clone 38, calbindin (NTTA #2), cryptic determinants (NTTA #6, 7), and NTTA #4 from an unknown βcell cDNA open reading frame referenced below.

“Autoantigen” or “target-determinant” as used herein is an antigen that either is the initiating antigen, i.e. the initial target of abnormal autoimmune response (also called the inducing antigen in the case of an induced animal model of autoimmune disease), or an antigen that, when autoimmunity spreads, becomes a target of autoimmune response, notably a target for Th1 T-cells (e.g. heat shock proteins).

“Bystander Antigen” is an antigen exposed to the immune system and present in an organ or tissue that is the target tissue of an autoimmune disease. Bystander antigens may but need not be autoantigens. When a bystander antigen is successfully used in active tolerization (e.g. when it is orally administered) it elicits regulatory T-cells, which home-in on the target tissue and abate autoimmune responses by secretion of various regulatory cytokines such as interleukin 4 (IL-4), interleukin 10 (IL-10), and transforming growth factor beta (TGF-β). Unlike NTTAs, however, some bystander antigens can become involved in abnormal autoimmune response. For example, proteolipid protein (PLP) is a bystander antigen in the animal model EAE (experimental acute encephalomyelitis) which is induced by immunization with myelin basic protein (MBP). However, PLP is not an NTTA because it can be the target of autoimmunity either in the MBP-induced animal model EAE (e.g. during later stages of the autoimmunity associated with the disease which is characterized by determinant spreading) or in the PLP-induced EAE model (wherein PLP itself is the initiating autoantigen), or in the actual human autoimmune disease multiple sclerosis (MS patient populations have been shown to have activated autoreactive T-cells that recognize PLP). Thus, NTTAs can be deemed a narrow subset of bystander antigens, and further can encompass determinants of bystander antigens not involved in autoimmunity.

“Autoimmune Disease” is a malfunction of the immune system, i.e., a pathological condition (or an induced or spontaneous animal model therefor) in which the immune system of a mammal (including a human) ceases or fails to recognize self (i.e. autologous antigens) and as a result treats these substances as if they were foreign antigens and mounts an immune response against them. Typically, autoimmune diseases that are organ- or tissue-specific involve, in whole or in part, priming or activation of autoreactive cells expressing the Th1 phenotype (rather than the Th2 phenotype, which is expressed by regulatory T-cells). Non-limiting examples of autoimmune diseases include multiple sclerosis (MS), Type 1 diabetes (IDDM), rheumatoid arthritis (RA), uveoretinitis (UR), and autoimmune thyroiditis (AT).

“Cryptic Determinant” or “Cryptic Antigen” means a peptidic segment of an antigen which can be recognized by T-cells of the immune system, but to which nevertheless the immune system mounts no response when a host (mammalian or human) is immunized with the entire antigen. However, if a peptide consisting essentially of a cryptic determinant is used to immunize a host, the host develops an immune response against that determinant. Sercarz, E. E., et al., Annu. Rev. Immunol., 11:729-766, 1993. Some cryptic determinants are NTTA and some are target determinants.

“Immune System Disorder” or “Abnormal Immune Response” includes without limitation: allergy, and abnormal autoimmune response associated with an autoimmune disease. It should be understood that because the present invention manipulates T cells and abates inflammatory responses by inducing regulatory responses, its applicability is not limited to autoimmune diseases but extends to allergy and indeed any other disorder involving inflammation of an organ or tissue. Such disorders include: Addison's disease, adult respiratory distress syndrome, allergies, anemia, asthma, atherosclerosis, bronchitis, cholecystitus, Crohn's disease, ulcerative colitis, atopic dermatitis, dermatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome and autoimmune thyroiditis; complications of cancer, hemodialysis, extracorporeal circulation; viral, bacterial, fungal, parasitic, protozoal and helminthic infections, trauma, and reperfusion injury.

“Treatment” means a preventive or a therapeutic regimen or both. “Prevention” shall include not only complete prevention of a disorder but also delay in onset of clinical symptoms or a reduction in the underlying abnormal immune response which results in a delay of clinical onset.

“Clinical Onset” of an autoimmune disease means the appearance of recognized clinical symptoms, such as persistent hyperglycemia or hypoinsulinemia in IDDM; joint swelling, joint inflammation, joint tenderness in rheumatoid arthritis; fatigue, paresthesis, nystagmus, weakness, cognitive dysfunction, peripheral neuropathy, oligoclonal bands and brain plaques detected by magnetic resonance imaging (MRI), and spasticity for multiple sclerosis; retinal inflammation, Behcet's disease, pars planitis, ocular sarcoid, birdshot retinochorioidopathy, sympathetic ophthalmia, and Vogt-Koyanagi-Harada syndrome in uveoretinitis; and goiter, hypothyroidism, increased PBI and increased RAI uptake in autoimmune thyroiditis (Hashimoto's thyroiditis).

“Pre-clinical symptoms” include the appearance of activated T-cells (Th1) at the target tissue; and immunity events such as the co-appearance of islet cell antibodies (alone or in the copresence of anti-insulin antibodies) in persons at significant risk for development of IDDM; optic neuritis or oligoclonal bands or brain plaques on MRI for multiple sclerosis; rheumatoid factor for rheumatoid arthritis; and increased PBI and/or autoantibodies in thyroiditis.

“Determinant” is a portion of an antigen recognized by T-cells specific to the antigen.

“Late stages of autoimmunity” means a stage in the development of autoimmunity after autoimmunity has spread to a more than one antigen, more specifically more than two antigens or even three or more antigens or even after clinical symptoms have appeared. It should be noted, however, that autoimmunity must still be ongoing to a substantial degree. In the case of IDDM patents, for example, a late stage of autoimmunity begins with determinant spreading to two or more antigens and ends with the substantial cessation of autoimmune response after approximately 90-95% of the islet cells have been destroyed. It should be noted, however, that the demarcation may be less clear-cut in other autoimmune diseases.

“Peptide” is any compound having a sequence consisting of amino acid residues. In the present invention, peptide NTTAs will have a sequence of at least 8-9 amino acids (at least 13-14 if class II restricted), and up to about 100 amino acids, preferably up to about 50, most preferably up to about 20.

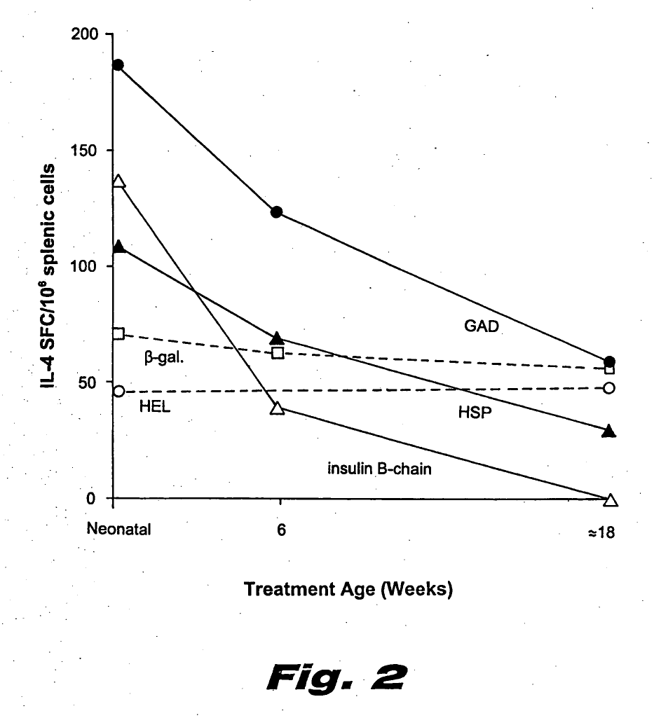

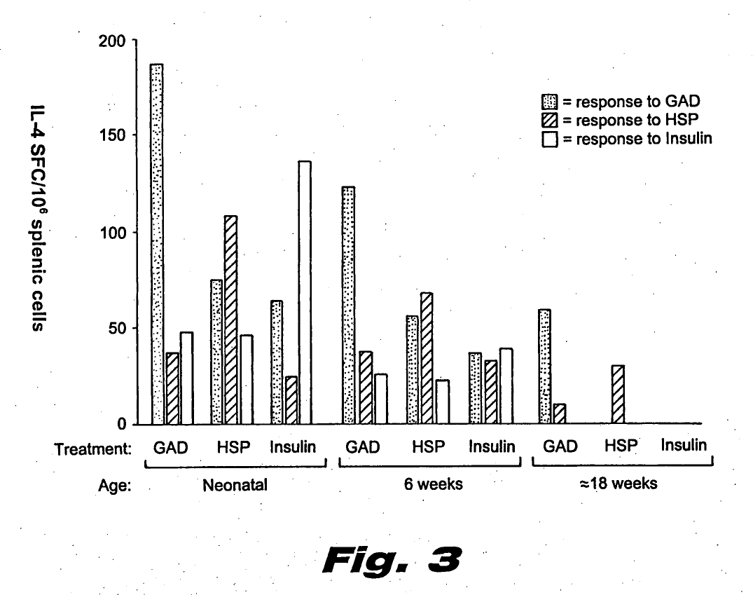

The present inventors have postulated that the efficacy of antigen-based immunotherapy depends in part on the availability of large T cell subpopulations that are available for induction towards regulatory responses. The inventors found that while the ability to prime Th2 responses to non-target tissue antigens is unaffected by the spontaneous disease process in NOD mice, the ability to prime Th2 responses to autoantigens declines substantially with disease progression (2, 17). Late in the disease process, treatment with some βCAAs could no longer promote Th2 spreading, or even prime detectable Th2 responses (FIGS. 2 and 3). This attenuation of inducible Th2 immunity to autoantigens indicates that the recruitment of autoantigen-reactive T cells into the spontaneous disease process depletes the pool of uncommitted autoantigen-reactive T cells that would otherwise be available for priming toward regulatory responses. Indeed, the inventors observed that at each stage of the disease process, autoantigens which primed greater antigen-specific Th2 responses tended to promote more extensive spreading of Th2 immunity towards other autoantigens. Notably, autoantigens which more effectively promoted Th2 immunity late in the disease process were better able to prolong the survival of transplanted syngeneic islets in diabetic NOD mice (2, 3).

The implication of these findings is that target tissue antigens which have large uncommitted T cell precursor pools late in the disease process are more effective immunoregulators and could therefore be used in antigen-based immunotherapies. The present inventors turned their attention to neglected target tissue antigens as candidates for such immunotherapy.

After extensive research, only about a dozen different antigens have been identified which become targets of autoimmune responses in rodent and human IDDM. (Even fewer autoantigens have been described in EAE or in EAU (experimental autoimmune uveoretinitis) or in induced models of rheumatoid arthritis. Apparently, the vast majority of β cell antigen determinants do not become targets of the autoimmune response (at least to detectable levels). However, it is clear that T cells that are potentially reactive against NTTAs are present in NOD mice, as immunization with NTTAs can elicit vigorous T cell responses. In addition, others have already identified GAD determinants that are ignored by the spontaneous autoimmune response but elicit recall responses after immunization with whole antigen (23). The inventors believe that for most β cell antigen determinants, negative selection has left only T cells which even in an inflammatory environment fail to interact with peptide/MHC complexes at levels sufficient for activation and expansion. Thus, there appears to be a hierarchy of β cell antigen determinants.

1) Primary determinants that are the first to be recognized by T cells during the initial loss of self-tolerance. The earliest β cell-reactive T cells which spontaneously activate and evade peripheral tolerance induction (due to the initially low levels of co-stimulatory factors) should be T cells with high avidity for βCAA determinants. (GADp35 may qualify as such a determinant).

2) Once a pro-inflammatory response takes root, it further promotes an inflammatory environment by cytokine production and the recruitment and activation of APC (antigen presenting cells). Consequently, lower avidity βCAA reactive T cells can interact with antigen/MHC complexes and co-stimulatory factors at above threshold levels for activation (insulin and HSP may fall in this category).

3) Finally, there are tertiary determinants that appear to be neglected by the autoimmune response. However, these determinants can be recognized by memory T cells which have been experimentally primed against these determinants.

NTTAs can be due either to “determinant crypticity” or to “repertoire crypticity” (24-26). Self-determinants that are efficiently presented should inactivate T cells with high and medium avidity for the determinant by central or peripheral tolerance induction mechanisms (24). Accordingly, only low avidity potentially reactive T cells remain, which may not reach threshold levels for activation during an autoimmune disease process. This lack of response to a strong self-determinant by a negatively selected T cell repertoire has been termed “repertoire crypticity” (26). Self-determinants may also be neglected because following the processing of whole antigen they are very inefficiently presented. These determinants make little impact on T cell selection such that a large repertoire of high to low avidity potentially reactive T cells are allowed to persist. However, even in the context of an inflammatory environment, these determinants are so weakly presented that they generally fail to activate native T cells. Such weak self-determinants that can be potentially recognized by an unselected T cell repertoire are the classic “cryptic determinants” defined by Sercarz (25), and constitute a subset of NTTAs.

The inventors reasoned that even in advanced stages of an autoimmune process, large uncommitted T cell pools should exist that would be specific to some target tissue antigen determinants which have been ignored by the spontaneous autoimmune response. The inventors set out to identify a panel of different β cell NTTAs and test whether they could provide more effective immunotherapeutics late in the disease process. The inventors theorized that neglected determinants should exist within β cell proteins that are not recognized by the spontaneous autoimmune process, as well as within autoantigens (GAD was selected as a model). The methods and rationale for selecting the various candidate NTTAs are detailed in Example 4 below.

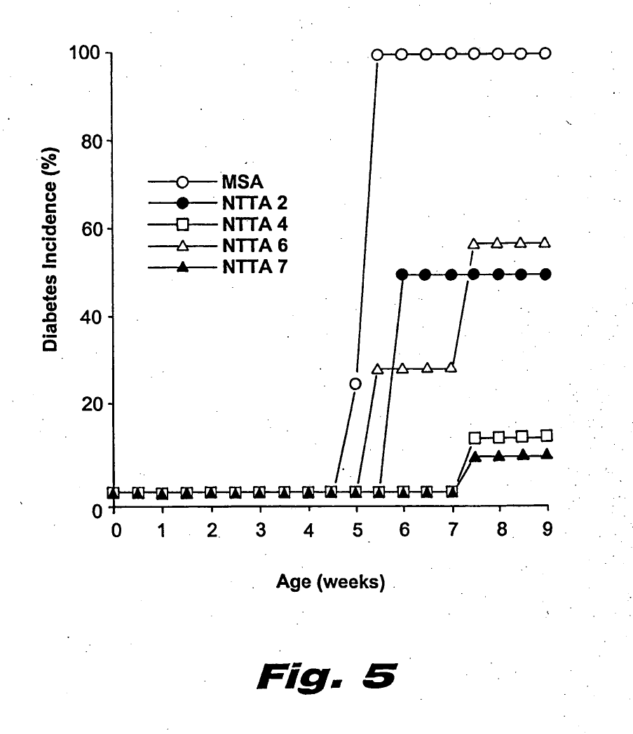

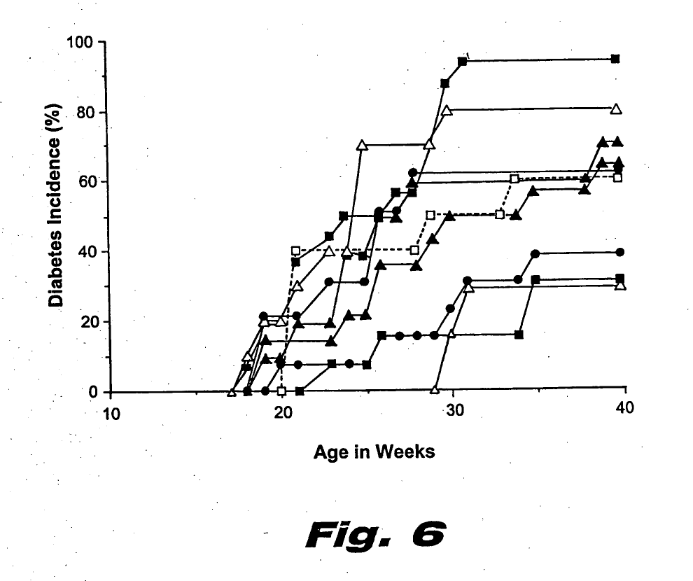

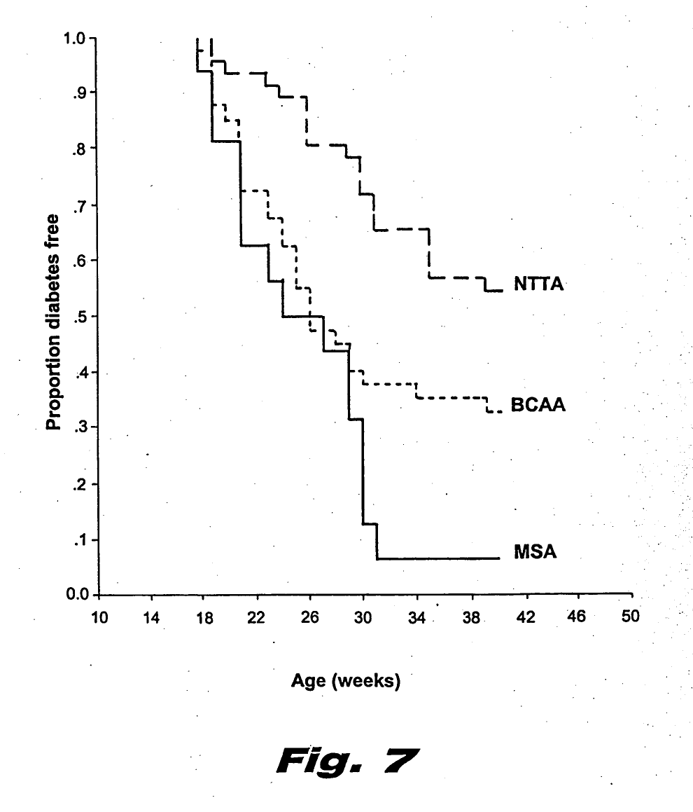

The ability of NTTA treatment to prevent the adoptive transfer of IDDM was tested. T cells from NTTA-treated NOD mice inhibited the adoptive transfer of IDDM (FIG. 5), demonstrating that NTTA administration induces regulatory responses which are adoptively transferable. The ability of NTTAs and βCAA target determinants to inhibit disease in NOD mice with advanced autoimmunity was compared. Treatment with any βCAA target determinant (insulin B-chain, HSP 277, GADp35, GADp34) provided only a non-significant trend toward protection when compared to mice treated with a control non-target tissue self-peptide or unmanipulated NOD mice (FIG. 6). In contrast, treatment with NTTAs significantly inhibited disease compared to control groups and groups treated with βCAA target determinants (FIGS. 6 and 7).

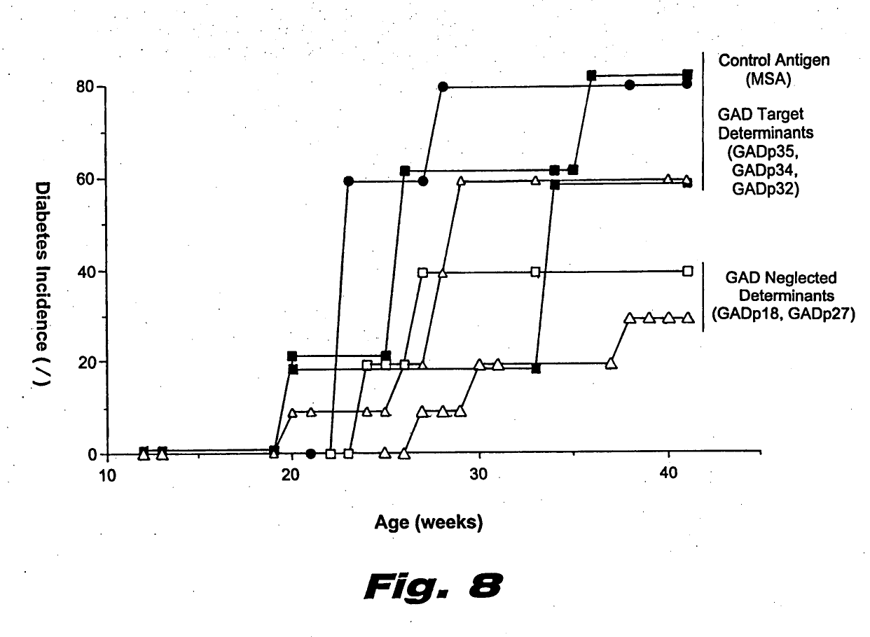

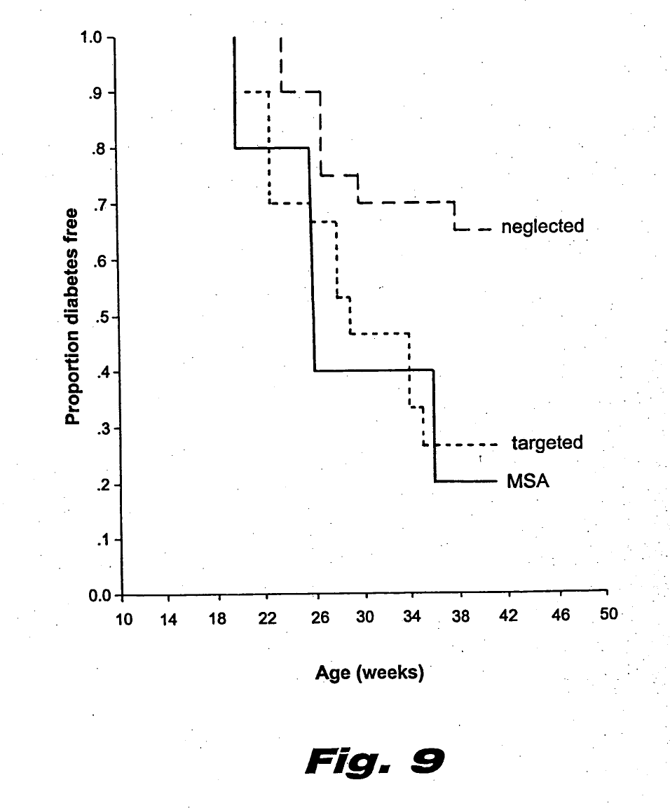

In another set of experiments, the therapeutic effect of administering GAD target determinants was compared to that of GAD neglected determinants. While peptides containing GAD target determinants have been shown to effectively prevent IDDM when given to newborn NOD mice (27), these treatments conferred no protection when administered at 12 weeks in age. In contrast, treatment with neglected GAD determinants conferred long-term protection from disease (FIGS. 8 and 9). The clear differences in the efficacy between treatments with βCAA target determinants and NTTAs strongly support the view that even late in autoimmunity uncommitted cells exist which cannot be marshaled by target antigens but are only available to be manipulated by NTTAs into expressing a regulatory phenotype.

While the existence of a low level of T cell responses to these NTTAs (below the level of detection), cannot be excluded, such low reactivity would not affect the principle of the present invention that large T cell precursor pools are available against NTTAs and that precursor availability is an important factor in determining the efficacy of an antigen-based immunotherapy. It should be noted that not all NTTAs are expected to have the same degree of efficacy.

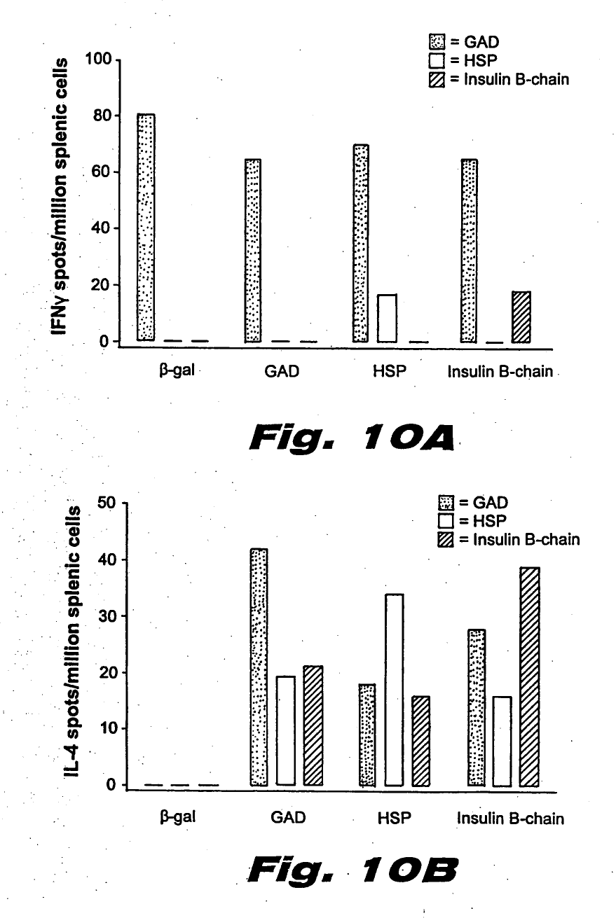

The inventors observed that NOD treated neonatally with insulin B-chain or HSP 277 in IFA displayed both Th2 and Th1 responses to the injected autoantigen at 4 weeks in age (FIG. 10). Normally, NOD mice do not develop detectable Th1 responses to insulin B-chain and HSP 277 until several weeks later. Treatment with βCAA target determinants primed accelerated Th1 responses to the injected antigen because partially primed T cells to the injected antigen were already present. While such T cells would not normally become fully activated until later in the disease process, when a more pro-inflammatory environment was established, the greatly increased presentation of the injected autoantigen on APC apparently drove some T cells that had been partially activated toward the Th1 phenotype to become fully activated and expand to levels of detectability. Thus, unlike non-target tissue antigens, the administration of autoantigens can prime accelerated pro-inflammatory responses to the injected antigen, consistent with the fear that autoantigen administration could exacerbate the disease process.

Based on the experiments detailed herein, like T cells reactive to antigens from tissues other than the target tissue, T cells reactive to NTTAs are not activated (at least to the level of detection)—however, they can be activated in the periphery via immunization. If partially primed T cells against NTTAs are not present in the islets, the administration of these antigens will prime only naive T cells in the periphery, which will be guided by the adjuvant towards a unipolar Th2 response—as observed after administration of self-MBP, MSA or foreign antigens. (Even if partially activated NTTA-reactive T cells are present in the islets, they would be present at much lower frequency than those reactive to target determinants, which supports the safety of administration of NTTAs). Thus, while the administration of target determinants (or altered peptide ligands thereof) has an inherent danger of boosting established pathogenic responses, the administration of NTTAs may circumvent this danger because of the ability to induce a polarized regulatory T cell response to NTTAs.

Although the present invention is being described by reference to autoimmune diseases, notably IDDM, the findings and conclusions can be extrapolated to other abnormal inflammatory immune responses regardless of the cause. The present invention involves manipulation of T cells, i.e., relief of a symptom, albeit one ubiquitously present in all inflammation. The present invention does not involve treatment (much less a cure) of underlying causes, which may be diverse.

Individuals who are identified as being at risk of developing IDDM (based on autoantibody screening), or are presenting the first clinical or preclinical symptoms of MS or RA, VA or AT are likely to already have an advanced disease process. The inventors have shown that βCAAs differ greatly in their ability to protect transplanted syngeneic islets in diabetic NOD mice and that there was a correlation between the ability of a βCAA treatment to induce regulatory immunity and its ability to prolong the survival of transplanted autoimmune disease progression, but that antigens from tissues other than the target tissue were able to induce vigorous responses at all stages of the NOD disease process. Thus, the inventors reasoned that large precursor pools might be available against some NTTAs and that, late in an autoimmune disease process, the administration of some NTTAs would be more capable of eliciting regulatory responses. Based on the present data, NTTA treatments are indeed more effective than treatments with βCAA target determinants, and may be safer than administering autoantigen target determinants.

### Antigens

The sequences of autoantigens and NTTAs employed herein (or capable of being employed) for IDDM and other autoimmune diseases or models thereof are either provided or referenced below. As stated above, NTTAs can be identified as whole antigens or portions of antigens. They can even be identified within autoantigens (e.g., as cryptic determinants of autoantigens).

Accordingly, this section provides: (a) antigens that have been already identified as NTTAs; (b) antigens expressed in tissue affected in an autoimmune disease (or allergy) for which autoimmunity has not been reported (these can be considered potential NTTAs and can be tested or peptide fragments of them can be tested); and (c) known autoantigens as potential sources for peptide NTTAs corresponding to determinants within said autoantigens that are not recognized by the autoimmune response.

IDDM

GAD: glutamate decarboxylase 65 (pancreatic islets and brain, 65 kD) GenBank Accession # NP_{—}000809 (this is a known autoantigen)

(SEQ ID #1)

MASPGSGFWSFGSEDGSGDSENPGTARAWCQVAQKTGGIGNKLCALLYGD
AEKPAESGGSQPPRAAARKAACACDQKPCSCSKVDVNYAFLHATDLLPAC
DGERPTLAFLQDVMNILLQYVVKSFDRSTKVIDFHYPNELLQEYNWELAD
QPQNLEEILMHCQTTLKYAIKTGHPRYFNQLSTGLDMVGLAADWLTSTAN
TNMFTYEIAPVFVLLEYVTLKKMREIIGWPGGSGDGIFSPGGAISNMYAM
MIARFKMFPEVKEKGMAALPRLIAFTSEHSHFSLKKGAAALGIGTDSVIL
IKCDERGKMIPSDLERRILEAKQKGFVPFLVSATAGTTVYGAFDPLLAVA
DICKKYKIWMHVDAAWGGGGLLMSRKHKWKLSGVERANSVTWNPHKMMGV
PLQCSALLVREEGLMQNCNQMHASYLFQQDKHYDLSYDTGDKALQCGRHV
DVFKLWLMWRAKGTTGFEAHVDKCLELAEYLYNIIKNREGYEMVFDGKPQ
HTNVCFWYIPPSLRTLEDNEERMSRLSKVAPVIKARMMEYGTTMVSYQPL
GDKVNFFRMVISNPAATHQDIDFLIEEIERLGQDLGKRNAVEVLKREPLN
YLPL.

Human glutamate decarboxylase, 67 kd isoform (GAD-67) (67 kD GLUTAMIC ACID DECARBOXYLASE) GenBank Accession # Q99259

(SEQ ID #2)

MASSTPSSSATSSNAGADPNTTNLRPTTYDTWCGVAHGCTRKLGLKICGF
LQRTNSLEEKSRLVSAFKERQSSKNLLSCENSDRDARFRRTETDFSNLFA
RDLLPAKNGEEQTVQFLLEVVDILLNYVRKTFDRSTKVLDFHHPHQLLEG
MEGFNLELSDHPESLEQILVDCRDTLKYGVRTGHPRFFNQLSTGLDIIGL
AGEWLTSTANTNMFTYEIAPVFVLMEQITLKKMREIVGWSSKDGDGIFSP
GGAISNMYSIMAARYKYFPEVKTKGMAAVPKLVLFTSEQSHYSIKKAGAA
LGFGTDNVILIKCNERGKIIPADFEAKILEAKQKGYVPFYVNATAGTTVY
GAFDPIQEIADICEKYNLWLHVDAAWGGGLLMSRKHRHKLNGIERANSVT
WNPHKMMGVLLQCSAILVKEKGILQGCNQMCAGYLFQPDKQYDVSYDTGD
KAIQCGRHVDIFKFWLMWKAKGTVGFENQINKCLELAEYLYAKIKNREEF
EMVFNGEPEHTNVCFWYIPQSKRGVPDSPQRREKLHKVAPKIKALMMESG
TTMVGYQPQGDKANFFRMVISNPAATQSDIDFLIEEIERLGQDL.

GAD p 18:

| | |
|---|---|
| PEVKEKGMAAVPRLIAFTSE. | (SEQ ID #3) |

GAD p 27:

| | |
|---|---|
| PLQCSALLVREEGLMQSCNQ. | (SEQ ID #4) |

Clone 38: P. Neophytou, et al., Diabetes, 45:127-133, 1996
Calbindin: Potential NTTA: No autoimmunity has been reported against this antigen. The inventors employed mouse calbindin isolated and elucidated by Nordquist, D. T. et al, J. Neunsci., 8: 4780 (1988).

Calbindin: Chain A, Solvated Refinement Of Ca-Loaded Calbindin D9k GenBank Accession # 1B1G

(SEQ ID #5)

MKXSPEELKGIFEKYAAKEGDPNQLSKEELKLLLQTEFPSLLKGGSTL.
DELFEELDKNGDGEVSFEEFQVLVKKISQ

Chain B, GenBank Accession # 1A03

(SEQ ID #6)

MASPLDQAIGLLIGIFHKYSGKEGDKHTLSKKELKELIQKELTIGSKL
QDAEIVKLMDDLDRNKDQEVNFQEYITFLGALAMIYNEALKG.

calbindin 2, (29 kD, calretinin) HUMAN GenBank Accession # NP_{—}001731.1

(SEQ ID #7)
MAGPQQQPPYLHLAELTASQFLEIWKHFDADGNGYIEGKELENFFQELEK
ARKGSGMMSKSDNFGEKMKEFMQKYDKNSDGKIEMAELAQILPTEENFLL
CFRQHVGSSAEFMEAWRKYDTDRSGYIEANELKGFLSDLLKKANRPYDEP
KLQEYTQTILRMFDLNGDGKLGLSEMSRLLPVQENFLLKFQGMKTSEEFN
AIFTFYDKDRSGYIDEHELDALLKDLYEKNKKEINIQQLTNYRKSVMSLA
EAGKLYRKDLEIVLCSEPPM.

calbindin 1 HUMAN GenBank Accession # AAD08724.1

(SEQ ID #8)

MAESHLQSSLITASQFFEIWLHFDADGSGYLEGKELQNLIQELQQARKKA
GLELSPEMKTFVDQYGQRDDGKIGIVELAHVLPTEENFLLLFRCQQLKSC
EEFMKTWRKYDTDHSGFIETEELKNFLKDLLEKANKTVDDTKLAEYTDLM
LKLFDSNNDGKLELTEMARLLPVQENFLLKFQGIKMCGKEFNKAFELYDQ
DGNGYIDENELDALLKDLCEKNKQDLDINNITTYKKNIMALSDGGKLYRT
DLALILCAGDN.

27 kDa calbindin HUMAN GenBank Accession # AAC62230.1

(SEQ ID #9)

MAESHLQSSLITASQFFEIWLHFDADGSGYLEGKELQNLIQELQQARKKA
GLELSPEMKTFVDQYGQRDDGKIG1VELAHVLPTEENFLLLFRCQQLKSC
EEFMKTWRKYDTDHSGFIETEELKNFLKDLLEKANKTVDDTKLAEYTDLM
LKLFDSNNDGKLELTEMARLLPVQENFLLKFQGIKMCGKEFNKAFELYDQ
DGNGYIDENELDALLKDLCEKNKQDLDINNITTYKKNIMALSDGGKLYRT
DLALILCAGDN.

NTTA #2:

(SEQ ID #10)

| | |
|---|---|
| ELKNFLKDLLEKANKTVDDT | (from calbindin). |

NTTA #4:

| | | |
|---|---|---|
| LIKPDRCHHCSVCDKC | (from clone 38). | (SEQ ID #11) |

islet amyloid polypeptide (IAPP): This is a potential NTTA: no autoimmunity reported.

HUMAN ISLET AMYLOID POLYPEPTIDE PRECURSOR (diabetes-associated peptide) (DAP) (AMYLIN) (INSULINOMA AMYLOID PEPTIDE) GenBank Accession # P10997

(SEQ ID #12)

MGILKLQVFLIVLSVALNHLKATPIESHQVEKRKCNTATCATQRLANFLV
HSSNNFGAILSSTNVGSNTYGKRNAVEVLKREPLNYLPL.

islet amyloid polypeptide precursor; AMYLIN Accession # NP_{—}000406.1

(SEQ ID #13)

MGILKLQVFLIVLSVALNHLKATPIESHQVEKRKCNTATCATQRLANFLV
HSSNNFGAILSSTNVGSNTY.

NTTA #6:

| | | |
|---|---|---|
| NHLRATPVRSGSNPQ | (from IAPP). | (SEQ ID #14) |

NTTA #7:

| | | |
|---|---|---|
| TQRLANFLVRSSNNL | (from IAPP). | (SEQ ID #15) |

Neuropeptide y: neuropeptide Y HUMAN Accession # NP_{—}000896.1

(SEQ ID #16)

MLGNKRLGLSGLTLALSLLVCLGALAEAYPSKPDNPGEDAPAEDMARYYS
ALRHYINLITRQRYGKRSSPETLISDLLMRESTENVPRTRLEDPAMW.

For other autoimmune diseases, the following antigens constitute (either actual or putative) NTTAs for humans or are suitable as sources of NTTA peptides:

Rheumatoid Arthritis

Many of the antigens given below have been reported as having some animal or human involvement in autoimmunity. Nevertheless, they are useful at least for “mining” NTTA peptides according to methods such as described in Example 4.
proteoglycans: (involved in human osteoarthritis) Cs-Szabo, G. et al., Arthritis Rheum. 4:1037, 1997.
cartilage oligomeric matrix protein: Larson, E. et al., Br. J. Dermatol., 36:1258, 1997.

decorin: decorin Human GenBank Accession # NP_{—}001911

(SEQ ID #17).

MKATIILLLLAQVSWAGPFQQRGLFDFMLEDEASGIGPEVPDDRDFEPSL
GPVCPFRCQCHLRVVQCSDLGLDKVPKDLPPDTTLLDLQNNKITEIKDGD
FKNLKNLHALILVNNKISKVSPGAFTPLVKLERLYLSKNQLKELPEKMPK
TLQELRAHENEITKVRKVTFNGLNQMIVIELGTNPLKSSGIENGAFQGMK
KLSYIRIADTNITSIPQGLPPSLTELHLDGNKISRVDAASLKGLNNLAKL
GLSFNSISAVDNGSLANTPHLRELHLDNNKLTRVPGGLAEHKYIQVVYLH
NNNISVVGSSDFCPPGHNTKKASYSGVSLFSNPVQYWEIQPSTFRCVYVR
SAIQLGNYK.

link protein: link protein precursor human Accession # AAC04311.1

(SEQ ID #18)

MKSLLLLVLISFCWADHHSDNYTVDHDRVIHIQAENGPRLLVEAEQAKVF
SRRGGNVTLPCKFYRDPTAFGSGTHKIRIKWTKLTSDYLKEVDVFVSMGY
HKKTYGGYHGRVFLKGGSDNDASLVITDLTLEDYGRYKCEVIEGLEDDTA
VVALDLQGVVFPYFPRLGRYNLNFHEAQQACLDQDAVIASFDQLYDAWRS
GLDWCNAGWLSDGSVQYPITKPREPCGGQNTVPGVRNYGFWDKDKSRYDV
FCFTSNFNGRFYYLIHPTKLTYDEAVQACLNDGAQIAKVGQIFAAWKLLG
YDRCDAGWLADGSVRYPISRPRRRCSPSEAAVRFVGFPDKKHKLYGVYCF
RAYN.

(See, A. Guerassimov, et al., J. Rheumatol., 24:959, 1997)

Human proteoglycan link protein precursor (cartilage link protein) (LP) human GenBank Accession # P10915

(SEQ ID #19)

MKSLLLLVLISICWADHLSDNYTLDHDRAIHIQAENGPHLLVEAEQAKVF
SHRGGNVTLPCKFYRDPTAFGSGIHKIRIKWTKLTSDYLKEVDVFVSMGY
HKKTYGGYQGRVFLKGGSDSDASLVITDLTLEDYGRYKCEVIEGLEDDTV
VVALDLQGVVFPYFPRLGRYNLNFHEAQQACLDQDAVIASFDQLYDAWRG
GLDWCNAGWLSDGSVQYPITKPREPCGGQNTVPGVRNYGFWDKDKSRYDV
FCFTSNFNGRFYYLIHPTKLTYDEAVQACLNDGAQIAKVGQIFAAWKILG
YDRCDAGWLADGSVRYPISRPRRRCSPTEAAVRFVGFPDKKHKLYGVYCF
RAYN.

Proteoglycan link protein precursor (CARTILAGE LINK PROTEIN) (LP) (CHICKEN) GenBank Accession # P07354

(SEQ ID #20)

MTSLLFLVLISVCWAEPHPDNSSLEHERIIHIQEENGPRLLVVAEQAKIF
SQRGGNVTLPCKFYHEHTSTAGSGTHKIRVKWTKLTSDYLKEVDVFVAMG
HHRKSYGKYQGRVFLRESSENDASLIITNIMLEDYGRYKCEVIEGLEDDT
AVVALNLEGVVFPYSPRLGRYNLNFHEAQQACLDQDSIIASFDQLYEAWR
SGLDWCNAGWLSDGSVQYPITKPREPCGGKNTVPGVRNYGFWDKERSRYD
VFCFTSNFNGRFYYLIHPTKLTYDEAVQACLKDGAQIAKVGQIFAAWKLL
GYDRCDAGWLADGSVRYPISRPRKRCSPNEAAVRFVGFPDKKHKLYGVYC
FRAYN.

link protein 2—RAT Accession # LKRT2

(SEQ ID #21)

DHLSDSYTPDQDRVIHIQAENGPRLLVEAEQAKVFSHRGGNVTLPCKFYR
DPTAFGSGIHKIRIKWTKLTSDYLREVDVFVSMGYHKKTYGGYQGRVFLK
GGSDNDASLIITDLTLEDYGRYKCEVIEGLEDDTAVVALELQGVVFPYFP
RLGRYNLNFHEARQACLDQDAVIASFDQLYDAWRGGLDWCNAGWLSDGSV
QYPITKPREPCGGQNTVPGVRNYGFWDKDKSRYDVFCFTSNFNGRFYYLI
HPTKLTYDEAVQACLNDGAQIAKVGQIFAAWKLLGYDRCDAGWLADGSVR
YPISRPWRRCSPTEAAVRFVGFPDKKHKLYGVYCFRAYN.

proteoglycan link protein precursor—HUMAN Accession # LKHU

(SEQ ID #22)

MKSLLLLVLISICWADHLSDNYTLDHDRAIHIQAENGPHLLVEAEQAKVF
SHRGGNVTLPCKFYRDPTAFGSGIHKIRIKWTKLTSDYLKEVDVFVSMGY
HKKTYGGYQGRVFLKGGSDSDASLVITDLTLEDYGRYKCEVIEGLEDDTV
VVALDLQGVVFPYFPRLGRYNLNFHEAQQACLDQDAVIASFDQLYDAWRG
GLDWCNAGWLSDGSVQYPITKPREPCGGQNTVPGVRNYGFWDKDKSRYDV
FCFTSNFNGRFYYLIHPTKLTYDEAVQACLNDGAQIAKVGQIFAAWKILG
YDRCDAGWLADGSVRYPISRPRRRCSPTEAAVRFVGFPDKKHKLYGVYCF
RAYN.

For additional potential NTTA antigens in rheumatoid arthritis see, generally, G. Ls-Szabo, et al., Arthritis Rheum., 40(6):1037, 1997 and Id, 38:660, 1995.
Uveoretinitis

rhodopsin: rhodopsin kinase HUMAN ACCESSION # NP_{—}002920

(SEQ ID #23)

MDFGSLETVVANSAFIAARGSFDGSSSQPSRDKKYLAKLKLPPLSKCESL
RDSLSLEFESVCLEQPIGKKLFQQFLQSAEKHLPALELWKDIEDYDTADN
DLQPQKAQTILAQYLDPQAKLFCSFLDEGIVAKFKEGPVEIQDGLFQPLL
QATLAHLGQAPFQEYLGSLYFLRFLQWKWLEAQPMGEDWFLDFRVLGKGG
FGEVSACQMKATGKLYACKKLNKKRLKKRKGYQGAMVEKKILMKVHSRFI
VSLAYAFETKADLCLVMTIMNGGDIRYHIYNVNEENPGFPEPRALFYTAQ
IICGLEHLHQRRIVYRDLKPENVLLDNDGNVRISDLGLAVELLDGQSKTK
GYAGTPGFMAPELLQGEEYDFSVDYFALGVTLYEMIAARGPFRARGEKVE
NKELKHRIISEPVKYPDKFSQASKDFCEALLEKDPEKRLGFRDETCDKLR
AHPLFKDLNWRQLEAGMLMPPFIPDSKTVYAKDIQDVGAFSTVKGVAFDK
TDTEFFQEFATGNCPIPWQEEMIETGIFGELNVWRSDGQMPDDMKGISGG
SSSSSKSGMCLVS

rhodopsin GenBank ACCESSION # AAD24751

(SEQ ID #24)

MNGTEGPAFYVPMSNATGVVRSPYEYPQYYLVAPWAYGLLAAYMFFLIIT
GFPVNFLTLYVTIEHKKLRTPLNYILLNLAIADLFMVFGGFTTTMYTSLH
GYFVFGRLGCNLEGFFATLGGEMGLWSLVVLAIERWMVVCKPVSNFRFGE
NHAIMGVAFTWVMACSCAVPPLVGWSRYIPEGMQCSCGVDYYTRTPGVNN
ESFVIYMFIVHFFIPLIVIFFCYGRLVCTVKEAAAQQQESETTQRAEREV
TRMVIIMVIAFLICWLPYAGVAWYIFTHQGSEFGPVFMTLPAFFAKTSAV
YNPCIYICMNKQFRHCMITTLCCGKNPFEEEEGASTTASKTEASSVSSSS
VSPA.

retinoid-binding protein irbp MOUSE GenBank Accession # AAA39331.2

(SEQ ID #25)

LVLSTLLWVPAGPTHLFQPSLVLDMAKILLDNYCFPENLMGMQAAIEQAM
KSHEILGISDPQTLAQVLTAGVQSSLSDPRLFISYEPSTLEAPQQAPVLT
NLTREELLAQIQRNIRHEVLEGNVGYLRVDDLPGQEVLSELGEFLVSHVW
RQLMSTSSLVLDLTHCSGGHFSGIPYVISYLHPGNTVMHVDTVYDRPSNT
TTEIWTLPEVLGERYSADKDVVVLTSGHTGGVAEDIAYILKQMRRAIVVG
ERTEGGALDLQKLRIGQSNFFLTVPVSRSLGPLGGGGQTWEGSGVLPCVG
TPAEQALEKALAILTRRALPGVVLRLQEALQDYYTLVDRVPGLLHHLASM
DYSAVVSEEDLVTKLNAGLQAVSEDPRLLVRATGPRDSSSRPETGPNESP
AATPEVPTEEDARRALVDSVFQVSVLPGNVGYLRFDRFADAAVLETLGPY
VLKQVWEPLQDTEHLIMDLRHPGGPSSAMPLVLSYFQGPEAGPVRLFTTY
DRRTNITQEHFSHRELLGQRYGNQRGVYLLTSHRTATAAEEFAFLMQSLG
WATLVGEITAGSLLHTCTVPLLDSPQGGLALTVPVLTFIDNHGEAWLGGG
VV.

interphotoreceptor retinoid-binding protein GALLUS GenBank Accession # AAD26334

(SEQ ID #26)

DMRFNIGGYTNWIPILCSYFFDAGHQVLLDKVYDRPSDSVKEIWTQPQLR
GERYGSQKGLIILTSAVTAGAAEEFVFIMKRLGRALIIGEQTSGGSHSPQ
TYXVD.

interphotoreceptor matrix proteoglycan 1 HUMAN Accession #NP 001554.1

(SEQ ID #27)

MYLETRRAIFVFWIFLQVQGTKDISINIYHSETKDIDNPPRNETTESTEK
MYKMSTMRRIFDLAKHRTKRSAFFPTGVKVCPQESMKQILDSLQAYYRLR
VCQEAVWEAYRIFLDRIPDTGEYQDWVSICQQETFCLFDIGKNFSNSQEH
LDLLQQRIKQRSFPDRKDEISAEKTLGEPGETIVISTDVANVSLGPFPLT
PDDTLLNEILDNTLNDTKMPTTERETEFAVLEEQRVELSVSLVNQKFKAE
LADSQSPYYQELAGKSQLQMQKIFKKLPGFKKIHVLGFRPKKEKDGSSST
EMQLTAIFKRHSAEAKSPASDLLSFDSNKIESEEVYHGTMEEDKQPEIYL
TATDLKRLISKALEEEQSLDVGTIQFTDEIAGSLPAFGPDTQSELPTSFA
VITEDATLSPELPPVEPQLETVDGAEHGLPDTSWSPPAMASTSLSEAPPF
FMASSIFSLTDQGTTDTMATDQTMLVPGLTIPTSDYSAISQLALGISHPP
ASSDDSRSSAGGEDMVRHLDEMDLSDTPAPSEVPELSEYVSVPDHFLEDT
TPVSALQYITTSSMTIAPKGRELVVFFSLRVANMAFSNDLFNKSSLEYRA
LEQQFTQLLVPYLRSNLTGFKQLEILNFRNGSVIVNSKMKFAKSVPYNLT
KAVHGVLEDFRSAAAQQLHLEIDSYSLNIEPADQADPCKFLACGEFAQCV
KNERTEEAECRCKPGYDSQGSLDGLEPGLCGLAQRNARSSRERELHAVPD
HSENQAYKTSVKSSKINKITR.

Retinal Phosphodiesterases

Additionally, target antigens such as S-antigen and interphotoreceptor retinoid-binding protein, upon fragmentation, are likely to yield peptide NTTAs such as peptides consisting of cryptic determinant peptides. Other retinal antigens for which no human autoimmunity has been reported are:

| Gene | Reference |
|---|---|
| Bestrophin | Nature Genetics 19: 241-247, 1998 |
| RP65 1q31 | Nature Genetics 17: 139-140 and 194-197, 1997 |
| TULP1 6p21.3 | Nature Genetics 18: 174-176, 1998 |
| PDEA 5q31.2-34 | Nature Genetics 11: 468-471, 1995 |
| PDEB 4p16.3 | PNAS 92: 3249-53, 1995 |
| Peripherin/RDS 6p | Nature 354: 480-83, 1991 |
| | Science 264: 1604-1608, 1994 |
| | Hum. Mutat. 10: 301-9, 1997 |
| CRALBP 15q26 | Nature Genetics 17: 198-200, 1997 |
| CRX 19q13.3 | Neuron 19: 1329-36, 1998 |
| | Nature Genetics 18: 311-12, 1998 |
| ROM-1 11q13 | Science 264: 1604-1608, 1994 |
| | IOVS 38: 1972-1982, 1997 |
| RETGC 17p13.1 | Nature Genetics 14: 461-462, 1996 |
| GNAT1 | Nature Genetics 13: 358-360, 1996 |
| GNAT2 1p13 | Genomics 17: 442-48, 1993 |
| TIGR 1q23-25 | Science 275: 668-670, 1997 |
| | Human Mol. Genet. 6: 2091-97, 1997 |
| TIMP-3 22q12.1 | Hum. Mol. Genet. 4: 2415-16 |
| CNCG 4p14 | PNAS 92: 10177-181, 1995 |
| RHOK 13q34 | Nature Genetics 15: 175-178, 1997 |

Multiple Sclerosis

Any myelin-associated enzyme other than cnpase is a potential NTTA since no autoimmunity has been reported. Such enzymes include without limitation:
Cholesterol ester hydrolase-3.1.1.13
Cholesterol ester synthetase-2.3.1.26
Testosterone 5α-reductase—Melcangi et al., Dev. Brain Res., 44:181 (1988).
UDP-galactose:ceramide galactosyltransferase-2.4.1.62
Cerebroside acyltransferase—Theret et al., Neurochem. Res., 14:1235 (1989).
Neuramimidase-3.2.1.18
CDP-ethanolamine: 1,2-diacylglycerolethanolaminephosphotransferase-2.7.8.1.
CDP-choline: 1,2-diacylglycerolcholinephosphotransferase-2.7.8.2
CTP:ethanolaminephosphate cytidylytransferase-2.7.7.14
Choline kinase-2.7.1.82
Ethanolamine kinase-2.7.1.32
Phosphatidylinositol-4,5-bisphosphate phosphodiesterase (phospholipase C)-3.1.4.11
Phosphatidate phosphatase (phosphatidic acid phosphatase)-3.1.3.4.
Phospholipase D (phosphatidylcholine choline phosphohydrolase)-3.1.4.4
Calmodulin-stimulated kinase—Sulakhe et al., Biochem. J, 186:469 (1980a); Biochemistry, 19:5363 (1980b).
MBP-phosphate phosphatase—E. Miyamoto and S. Kakiuchi, Biochem. Biophys. Acta, 384:458 (1975).
Leucine aminopeptidase—N. L. Banik and A. N. Davidson, Biochem. J., 115:1051 (1969).
Culpain (CANP)—Sato et al., J. Neurochem., 39:97 (1982).
Metalloproteinase—Chantry, et al., J. Biol. Chem., 264:21603 (1989).
Proteolipid protein acyltransferase (autocatalysis)—Ross and Braun, J. Neurosci. Res., 21:35 (1988).
Acyl-proteolipid protein esterase—Bizzozero, O. A., Trans. Am. Soc. Neurochem., 22:265 (1991).
Carbonic anhydrase (carbonic hydrolase)-4.2.1.1

Other such myelin-associated proteins are disclosed in Martenson, R. E., Myelin: Biology and Chemistry, CRC Press 1992, pp. 532-534.

myelin basic protein HUMAN GenBank Accession # NP 002376.1 (This is involved in autoimmunity)

(SEQ ID #28)

MASQKRPSQRHGSKYLATASTMDHARHGFLPRHRDTGILDSIGRFFGGDR
GAPKRGSGKVPWLKPGRSPLPSHARSQPGLCNMYKDSHHPARTAHYGSLP
QKSHGRTQDENPVVHFFKNIVTPRTPPPSQGKGAEGQRPGFGYGGRASDY
KSAHKGFKGVDAQGTLSKIFKLGGRDSRSGSPMARR.

oligodendrocyte myelin glycoprotein HUMAN GenBank Accession # NP_{—}002535.1 (This is involved in autoimmunity)

(SEQ ID #29)

MSLCLFILLFLTPXILCICPLQCICTERHRHVDCSGRNLSTLPSGLQENI
IHLNLSYNHFTDLHNQLTQYTNLRTLDISNNRLESLPAHLPRSLWNMSAA
NNNIKLLDKSDTAYQWNLKYLDVSKNMLEKVVLIKNTLRSLEVLNLSSNK
LWTVPTNMPSKLHIVDLSNNSLTQILPGTLINLTNLTHLYLHNNKFTFIP
DQSFDQLFQLQEITLYNNRWSCDHKQNITYLLKWMMETKAHVIGTPCSTQ
ISSLKEHNMYPTPSGFTSSLFTVSGMQTVDTINSLSVVTQPKVTKIPKQY
RTKETTFGATLSKDTTFTSTDKAFVPYPEDTSTETINSHEAAAATLTIHL
QDGMVTNTSLTSSTKSSPTPMTLSITSGMPNNFSEMPQQSTTLNLWREET
TTNVKTPLPSVANAWKVNASFLLLLNVVVMLAV.

Proteolipid protein (This is also involved in autoimmunity)
   Allergy

The following are non-limiting examples of Antigens expressed in epithelial tissues, e.g., skin mucosa etc., allergens that can be probed for cryptic determinants and other NTTAs:

### Identification of NTTAs

NTTAs can be obtained or identified, as follows:

Lymphocytes are obtained from patients' (or animals') blood and are exposed to an antigen expressed in the organ or tissue of interest. The incubant is then probed for T- or B-cell proliferation, in accordance with any well-known technique. If there is neither T-cell nor B-cell proliferation, the antigen is a putative NTTA. The NTTA character can be confirmed by a simple proliferation assay. For example, lymphocyte proliferation can be measured by a tritiated thymidine incorporation assay [Bradley et al., In Selected Methods in Cellular Immunology, Mishell, B. B. and Shiigi, S. M., eds., W.H. Freeman and Company: San Francisco, p. 164 (1980)]. Alternatively, color formation of a metabolic dye, such as a tetrazolium compound like MTS (Promega) or MTT, can be used to evaluate proliferation [Gerlier D, et al., J Immunol Methods. 94:57-63 (1986); Denizot F, et al., J Immunol Methods. 89:271-7 (1986); Heeg K, et al., J Immunol Methods. 77:237-46 (1985)].

Alternatively, the Elisa Spot (“Elispot”) assay can be used to detect lymphocyte activation (as described, e.g., in U.S. Pat. No. 5,843,426, U.S. Pat. No. 5,750,356 and T. Forsthuber, et al., Science, 271:1728, 1996 and Surcel, et al., Immunology, 81:171, 1994) by detecting antibodies or increased levels of cytokine production (such as TNF and/or IFN-γ). Low-frequency human T lymphocytes can be detected using ELISPOT [McCutcheon et al. J Immunol Methods 210:149-66 (1997)]. Various cytokines, such as interleukin-2, interleukin-10, gamma interferon, [Sarawar and Doherty, J Virol 68:3112-9 (1994)] and interleukin-4 [EI Ghasali et al., Eur J Immunol 23:2740-5 (1993)] can be detected simultaneously with ELISPOT.

Peptides or peptidic segments of target antigens that constitute NTTA can be identified in a similar manner, but using the well-known overlapping peptide screening method (by which antigen fragments 20-40 amino acids in length are first synthesized and then used to stimulate T cells from patients as described, e.g., in Walden, Curr. Opin. Immunol., 8:68, 1996) in order to pinpoint the segments that constitute NTTA The NTTA peptides can be extracted from an autoantigen.

Alternatively, the method of Example 4 can be employed: the sequence of the entire antigen can be searched for the presence of motifs that are known or that are believed to be recognized by the TCR/MHC complex of the host to be treated. (See, reference 54 below and Wucherpfenig K. W. et al., J. Clin. Invest., 1997, 100: 1114; and Steinman L., Behring. Inst. Mitt, 1994, 94: 148). Peptides are then constructed incorporating such motifs, and tested as to whether they are targets for autoimmunity (or allergic reaction or inflammation as the case may be). This can substantially simplify the process of identifying a NTTA.

When employing whole antigens as NTTA, it is preferred to use tissue-specific antigens. To the extent that NTTAs disclosed (described or referenced) above are not tissue-specific, but are expressed in tissues other than the target organ (e.g., pancreas in IDDM), they could, in principle, be employed in treatment of other autoimmune diseases as well and indeed in the treatment of inflammation (e.g., NTTA fragments of heat shock protein).

It should be noted that NTTAs can be host species-specific. Accordingly, it is possible that a substance that is an NTTA in humans may be a target antigen in mice and vice versa. However, there are also determinants that are shared by two or more species, and NTTAs may be among them.

An antigen qualifies as an NTTA even if, in the future, detection limits for immune response become lower and reveal a very small amount of immunity against NTTA to be present. Example 11 relates to this topic by showing that the detection limit of the present technique is quite low.

Peptide NTTAs are preferred as tolerogens. A peptide NTTA should have a sequence of at least 8-9 (for class I restricted presentation) and at least 13-14 (for class II restricted presentation) amino acid residues, and should embody the entire neglected determinant. Additional amino acid residues can be included if they do not perturb the structure of the determinant so that it loses all or part of its ability to elicit regulatory responses. Thus peptides useful in the present invention may be said to “consist essentially” of neglected target determinants even if they also include such additional residues. However, the present invention also contemplates peptidic constructs in which more than one peptide NTTAs are joined with another substance or molecule or with one another.

### Mode and Dosage of Administration

Once identified, NTTAs can be administered to patients in amounts broadly ranging from 0.01 μg to 1000 mg per day in one or multiple divided doses, preferably 0.01 μg to 100 mg and most preferably 0.01 μg-10 mg. The mode of administration should be tolerogenic, i.e., conducive to induction of regulatory tolerance, (e.g., elicitation of Th2 responses). Thus, the NTTAs can be administered by oral, enteral, buccal, or nasal route (more generally, mucosal route), or by subcutaneous, intramuscular, or subdermal route, using no adjuvants or non-exacerbating adjuvants (such as alum), or using DNA vectors encoding the NTTAs.

The frequency of administration can be daily, or three times weekly, or less often e.g., in a pulse or bolus mode, or in a vaccine form according to the mode of administration.

The amount administered, and frequency of administration, will depend on the type and stage of the treated disease, the activity of the particular NTTA employed, the weight, age, and physical condition of the patient, and the method of administration and is thus subject to routine optimization.

The duration of the therapy can be as needed, and may continue indefinitely as long as benefits persist, as is within the skill of the art.

Oral pharmaceutical formulations within the present invention may contain inert constituents including pharmaceutically acceptable carriers, diluents, fillers, solubilizing or emulsifying agents and salts of the type that are well-known in the art. For example, tablets and caplets may be formulated in accordance with conventional procedures employing solid carriers, such as starch and bentonite, as is well-known in the art. Examples of solid carriers include bentonite, silica, dextrose and other commonly used carriers. Further non-limiting examples of carriers and diluents which may be used in the formulations of the present invention include saline and any physiologically buffered saline solution such as phosphate buffered saline, pH 7-8 and water.

Capsules containing NTTAs may be made from any pharmaceutically acceptable material, e.g, gelatin or a cellulose derivative. NTTAs may be administered in the form of sustained release oral delivery systems and/or enteric coated oral dosage forms, such as is described in U.S. Pat. No. 4,704,292 issued Nov. 3, 1987, U.S. Pat. No. 4,309,404 issued Jan. 5, 1982, or U.S. Pat. No. 4,309,406 issued Jan. 5, 1982.

The amount of NTTA contained in an individual oral dose need not in itself constitute an effective amount for suppressing immune response, since the necessary effective amount can be reached by administration of more than one dose. NTTAs may be administered daily or 2× or 3× weekly for at least three months, and the therapy may continue as long as benefits persist. Generally, oral administration of NTTAs will require higher doses (roughly by an order of magnitude) than other mucosal or parenteral modes of administration of NTTAs. Thus, oral doses will be within the range of 100 μg to 1000 mg (preferably 1001 g-200 mg).

Benefits can be assessed in various ways common in the art, such as a reduction in the number of activated autoreactive T cells, a reduction in Th1 cytokine production, a reduction in inflammation, a substantial prolongation of the time until clinical symptoms appear, an amelioration in clinical or preclinical symptoms or an arrestation of clinical or preclinical symptom progression.

For parenteral (e.g., i.p. or subcutaneous, intramuscular, subdermal) nonmucosal administration, the NTTAs can be incorporated into a physiologically acceptable solution or suspension. These preparations preferably contain from about 10 ng of NTTA to about 10 mg with 1 mg being a typical dose. Administration may occur once with a booster two weeks later, with a periodic repeating of vaccination (e.g. seasonally for allergies) being contemplated. An adjuvant or carrier may be included. DNA vaccines or gene therapy are also contemplated, in the manner described, e.g., by Waisman, A. et al, Nat. Med., 1996, 2: 899; or Kan-Mitchell, J. et al., Cancer Immunol. Immunother., 1993, 37: 15.

The solutions or suspensions can also include the following components: a sterile diluent such as, for example, water for injection, saline solution, fixed oils, polyethylene glycols, glycerine, propylene glycol, other synthetic solvents, and the like; antibacterial agents such as, for example, benzyl alcohol, methyl parabens, and the like; antioxidants such as, for example, ascorbic acid, sodium bisulfite, and the like; chelating agents such as, for example, ethylenediamine tetraacetic acid and the like; buffers such as, for example, acetates; citrates, phosphates, and the like, and agents for the adjustment of toxicity such as, for example, sodium chloride, dextrose, and the like. If an adjuvant is used, it should be a nonexacerbating adjuvant (e.g. alum).

The parenteral multiple dose vials can be of glass or plastic materials.

In mucosal administration, the dosages are expected to be generally the same as in s.c., i.m. or s.d. oral administration, except for inhalable dosage forms. The NTTA is placed in contact with the buccal, nasal, bronchial or pulmonary mucosa. Formulations useful for mucosal administration include those suitable for administration of polypeptides across the mucosal membrane. For example, U.S. Pat. Nos. 4,226,848 and 4,690,683 describe polymeric matrices useful in administering pharmaceuticals into the nasal cavity. U.S. Pat. No. 4,952,560 discloses an ointment formulation comprising a water-soluble protein and a monohydric alcohol which may be suitable for use in administering the present invention because it increases absorption of drugs across epithelial barriers. Methods of improving transcutaneous absorption of materials is described in U.S. Pat. No. 4,272,516. Each of these formulations and others well known in the art may be used for mucosal delivery of bystander antigen as described in the present invention.

Additional suitable formulations include commercially available vehicles and formulations which may but need not include surface active agents and other skin penetrants as absorption promoters. Specifically, U.S. Pat. No. 5,407,911 describes the use of axacycloalkane derivatives as absorption promoters for high molecular weight polypeptides. U.S. Pat. No. 5,397,771 describes the use of n-glycofurols in methods of administering pharmaceutical compositions across the mucosal membrane. Additionally, U.S. Pat. No. 4,548,922 discloses the use of water-soluble amphophilic steroids to increase absorption. Gel-based compositions, such as those described in Morimoto et al. (Chem. Pharm. Bull. 35(7):3041-3044) are also suitable for the present invention.

Where the NTTA is administered mucosally by inhalation, the quantity of peptide administered in, e.g., an aerosol dosage form by inhalation, is preferably between about 0.005 mg and 200 mg per dose, preferably between 0.01 mg-50 mg. The by-inhalation forms of the present invention may be administered to a patient in a single dosage form or multiple dosage forms. The exact amount to be administered may vary depending on the state and severity of any disease to be treated, the activity of the patient's immune system and the physical condition of the patient, and is subject to optimization.

Inhalable aerosol or spray pharmaceutical formulations may include, as optional ingredients, pharmaceutically acceptable carriers, diluents, solubilizing or emulsifying agents, and salts of the type that are well-known in the art. Specific non-limiting examples of the carriers and/or diluents that are useful in the aerosol pharmaceutical formulations of the present invention include water, normal saline and physiologically-acceptable buffered saline solutions such as phosphate buffered saline solutions, pH 7.0-8.0.

Examples of useful solubilizing and emulsifying agents are physiologically balanced salt solutions, phosphate buffered saline and isotonic saline. The salts that may be employed in preparing mucosal dosage forms of the invention include the pharmaceutically acceptable salts of sodium and potassium.

Aerosol compositions can be administered, e.g., as a dry powder or preferably in a finely divided aqueous solution phase. Preferred aerosol pharmaceutical formulations may comprise, for example, a physiologically-acceptable buffered saline solution.

Dry aerosol in the form of finely divided solid particles that are not dissolved or suspended in a liquid are also useful in the practice of the present invention. The compositions used in the present invention may be in the form of dusting powders and comprise finely divided particles having an average particle size of between about 1 and 5 microns, preferably between 2 and 3 microns. Finely divided particles may be prepared by pulverization and screen filtration using conventional techniques that are well known to those skilled in the art. The particles may be administered by inhaling a predetermined quantity of the finely divided material, which can be in the form of a dry atomized powder. Nebulizers or inhalers can be used to effect administration, as is well-known in the art.

Adjuvants (such as alum) can be added, if desired, and so can carriers, as is well-known in the art. Regulatory cytokines such as IL-4 and IL-10 can also be added.

The present invention is illustrated by the following examples, which are intended to illustrate the invention without limiting its scope.

### Methods

Mice: NOD mice were purchased from Taconic Farms (Germantown, N.Y.) and bred under specific pathogen-free conditions. Only female NOD mice were used in this study. In this NOD colony, insulitis begins at 4 weeks of age. The average age of disease onset is at 22 weeks, with about 80% of the mice displaying IDDM by 30 weeks of age. IDDM is anemia by repeat hyperglycemia.

Antigens. Mouse GAD65 (Lee, et al, Biochem. Biophys. Acta, 1216:157-160, 1993) and control Escherichia coli β-galactosidase were purified as described in Kaufman, D., et al., Nature 36:69, 1993. The various peptides were synthesized by standard fluorenyl methyloxycarbonyl (Fmoc) chemistry and purified by chromatography [TRUE?]. Control hen egg white lysozyme peptide HEL, immunogenic in NOD mice, was an academic gift but is commercially available. The amino acid composition of each peptide was verified by mass spectrometry. Insulin B-chain was purchased from Sigma.

Assays: At various times specified below, NOD mice received a single intraperitoneal (i.p.) injection of 100 μg of β-galactosidase (or other specified antigen) in 50% IFA (Gibco BRL, Gaithersburg, Md.). Four weeks later, serum samples were tested for antibodies to antigen and β-galactosidase by ELISA. β-Galactosidase or antigen (commercially available or recombined or synthesized) at 10 μg/ml was bound to 96-well plates (Nunc, Ruskilde, Denmark), in 0.1 M NaHCO, pH 9.6 (β-galactosidase) or pH 8.5 (GAD 65) at 4° C. overnight. The wells were rinsed with PBS and then blocked with 3% BSA in PBS for 1 hour. Mouse sera was added (0.1 ml of a 1/500 dilution) and incubated 1 hour at 37° C. Following washing, bound immunoglobulin was characterized using affinity-purified horseradish peroxidase (HRP)-coupled goat anti-mouse IgG+A+M (H+ L) (Pierce, Rockford, Ill.), or HRP-coupled goat anti-mouse isotype specific antibodies for IgG1 and IgG2a (Southern Biotech Associates, Birmingham, Ala.) and 2,2′-azino-bis(3-ethylbenzthiazoline-6-sulfonic acid) (ABTS). Serum samples from untreated BALB/c and AKR mice were used as negative controls.

ELISPOT analysis. At a specified time, mice received a single i.p. injection of 100 μg β-galactosidase or GAD65 or other specified antigen in 50% IFA. Fourteen days later, splenic T-cells were isolated and the frequency of β-galactosidase and GAD65-specific T-cells secreting IL-4, IL-5 and IFN-γ was determined by using the ELISPOT technique as described (Forsthuber, T., et al., Science, 271:1728, 1996), with the exception that the treating antigen and β-galactosidase (100 μg/ml) were used as antigens, and 11B11 together with biotinylated BVD6-24G2 (PharMingen, San Diego, Calif.) was used for capture and detection of IL-4. The resulting spots were counted manually.

Adoptive transfer of diabetes. At a specified time, NOD mice were injected i.p. with 100 μg GAD65 (or other specified antigen) or control β-galactosidase in 100 μl of 50% IFA and reinjected at 14 weeks of age. Five weeks later, single-cell suspensions of splenic mononuclear cells were prepared from each group, as well as from unmanipulated diabetic NOD mice. Ten million splenic mononuclear cells from the unmanipulated diabetic mice were mixed with an equal number of splenic mononuclear cells from GAD65 β-galactosidase-treated mice and injected intravenously into 5-week-old female NOD mice that had received 500 rad-γ-irradiation. Another control group received 1×10 splenic mononuclear cells obtained only from unmanipulated diabetic mice.

T-cell proliferation assays. Female NOD mice were injected i.p. at 8 weeks of age with 100 μg antigen, or control β-galactosidase, in 100 μl of 50% IFA. The mice were reinjected 2 weeks later. At 12 weeks of age, splenic T-cells were tested for proliferative responses to GAD65, hsp277, β-galactosidase and the HEL peptide, as previously described.

IDDM incidence. At specified times, groups of 10 female NOD mice were injected i.p. with 50 μg antigen or control β-galactosidase in 100 μl of 50% IFA. Another control group received 100 μl of 50% IFA alone. Because there may be a requirement for continual antigen presentation, the mice were reinjected every 6 weeks until 40 weeks of age. Urine glucose levels were monitored weekly for diabetes by Tes-tape (Lilly, Indianapolis, Ind.). After we observed abnormal glucose in the urine, blood glucose levels were monitored twice weekly. A recording of two consecutive blood glucose levels of greater than 13 mmol/l was considered as IDDM onset.

Transplantation of islets. Female NOD mice were monitored for the onset of IDDM, after which the mice were maintained on 1.0 to 1.5 units insulin (Humulin U, Lilly) per day. At the time of IDDM onset, mice were injected with either 100 μg of GAD65, hsp277, insulin B-chain or control β-galactosidase i.p. in 50% IFA. Ten days later, the mice were reinjected. Ten days after the second treatment, 3000 freshly isolated islets from newborn NOD mice were transplanted into the space beneath the kidney capsule, and Humulin administration was discontinued. The mice were reinjected every two weeks. Recurrence of diabetes is defined as two consecutive blood glucose levels of greater than 13 mmol/l.

### EXAMPLE 1

### A Th1 Amplificatory Cascade is Associated with Disease Progression

The involvement of Th1 and Th2 cells in both the spontaneous autoimmune process and in tolerance states has been difficult to address in non-transgenic mice primarily because of the very low frequency of autoreactive T cells within the T cell pool. Using an ELISPOT assay capable of characterizing T cells at the single cell level (Forsthuber et al, supra, 1996), we examined the natural development of B cell autoimmunity in NOD mice. When unmanipulated NOD mice were tested at the onset of insulitis (4 weeks of age), we detected vigorous IFNγ, but no IL-4 or IL-5 specific T cell responses to a single determinant of GAD (GAD p35) consistent with a unipolar Th1 response (2, 17). By 12 weeks of age, T cell autoimmunity had spread intramolecularly to additional GAD determinants and intermolecularly to insulin B-chain and HSP; all of these secondary autoimmune process are characterized by the spreading of unipolar Th1 type anti-βCAA reactivity.

### EXAMPLE 2

### Autoantigens Differ in Their Ability to Protect Transplanted Syngeneic Islets Grant in Diabetic NOD Mice

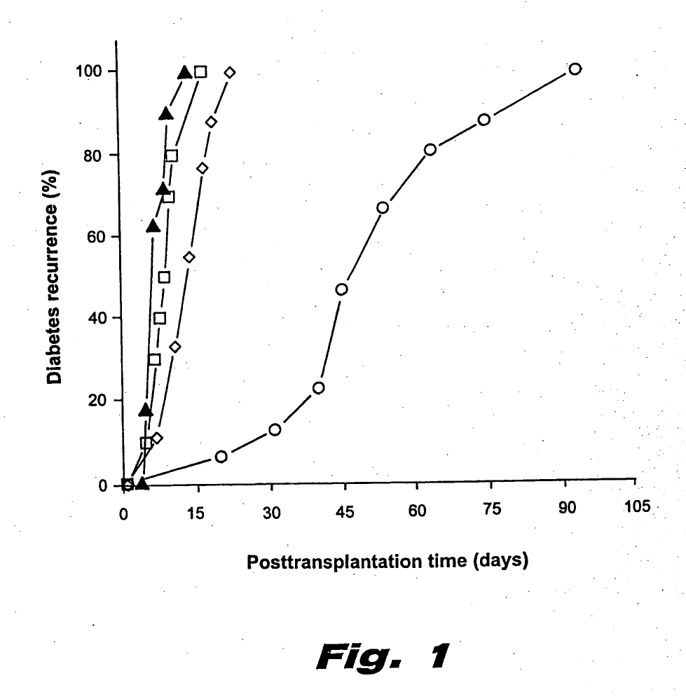

Based on the ability of autoantigen treatment to inhibit disease in pre-diabetic NOD mice with (1) we tested whether this treatment could also protect transplanted syngeneic β cells from the established autoimmune responses in diabetic NOD mice. Islets were transplanted into diabetic NOD mice that had been treated with β-galactosidase. Administering GAD/IFA intraperitoneally to recipient diabetic NOD mice prior to transplantation greatly extended syngeneic islet graft survival. While HSP peptide (HSPp277) treatment conferred a non-significant trend toward protection, insulin B-chain treatment lacked any protective effect. The results are shown in FIG. 1, wherein data are presented as time post-transplantation at which hyperglycemia recurred. These findings correlate with the extent to which each autoantigen was able to promote Th2 immunity in newly hyperglycemic animals (see Example 3, below).

### EXAMPLE 3

### Attenuation of Inducible Th2 Immunity with Disease Progression

Splenic T cells from NOD mice which had been treated at birth, 6 weeks in age, or at the onset of hyperglycemia (=118 weeks in age) with control foreign antigens or βCAAs were isolated and the frequency of T cells secreting IL-4 in response to the injected antigen was determined by ELISPOT. The data are represented as the mean number of IL-4 secreting spot forming colonies (SFC) per million splenic T cells. A similar pattern was observed for IL-5 secreting antigen-reactive T cells. The results are shown in FIG. 2.

Splenic T cells from mice treated at different stages of the disease process with control antigens or βCAAs were tested for antigen induced IL-4 and IL-5 T cell responses by ELISPOT. The data are represented as the mean number of IL-4 secreting SFC per million splenic T cells. The results are shown in FIG. 3. A similar pattern was observed for IL-5 secreting antigen-reactive T cells. In each case, non-target tissue antigens primed vigorous Th2 responses to the injected antigen, which however failed to spread to βCAAs (data not shown).

In brief the foregoing experiments show that: 1) responses to control non-target tissue antigens were unaffected by the disease process; 2) administration of βCAAs early in the disease process induced vigorous Th2 responses to the injected antigen which broadly spread to other βCAAs; 3) βCAAs which primed greater Th2 responses promoted more extensive spreading of Th2 immunity to other unrelated βCAAs (FIG. 2) and; 4) with disease progression, there was a steady decline in the ability of each βCAA to prime Th2 responses and Th2 spreading, suggesting that the uncommitted βCAA-reactive T cell pool that was available for priming was gradually shrinking (FIGS. 2 and 3). There was a correlation between the ability of different βCAAs to induce Th2 immunity late in the disease process and the success of these treatments to protect transplanted syngeneic β cells in diabetic NOD mice. This confirms that the decline of the ability to induce active tolerance to beta cell autoantigen with disease progression is not due to an intrinsic loss of immunologic responsiveness, but is consistent with depletion of the ranks of T cells that recognize the autoantigen but that remain uncommitted in terms of expressing an inflammatory or regulatory phenotype. Put differently, induction of active tolerance is a numbers game: the fewer the available T cells, the less effective the active tolerization.

### EXAMPLE 4

### Identification of β Cell NTTAs

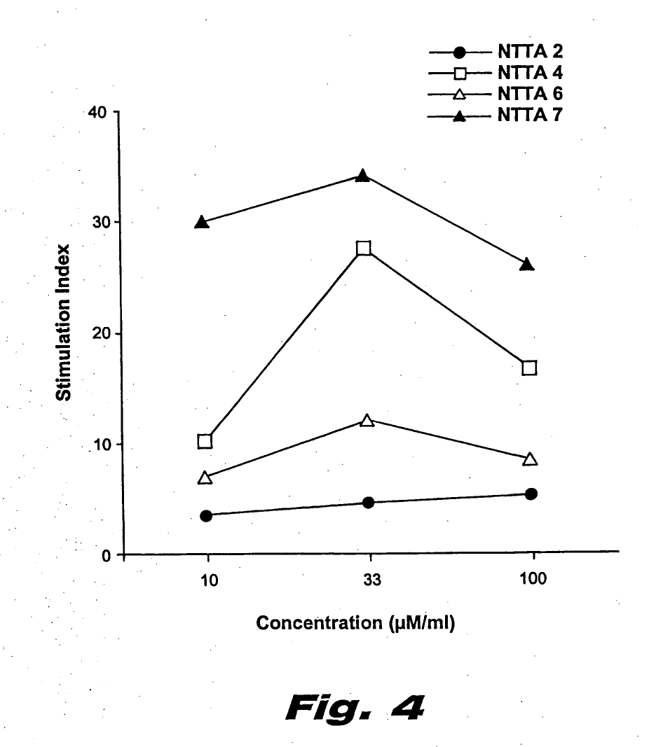

Transgenic animal models have shown that neoantigens which are expressed at low levels in peripheral tissues often have little impact on T cell education and elicit strong immune responses after immunization (4-8). Identifying NTTAs that were specifically expressed in the β cells at low levels was therefore preferred. The DNA sequence banks were screened for cDNA sequences isolated from mouse β cell subtraction libraries. Mouse cDNA sequences were selected from both known and unknown β cell cDNAs and from both β-cell specific and ubiquitous antigens, some of which appeared to be expressed only at low levels specifically in β cells. Other cDNAs that were expressed at higher levels in the β cells, or in other tissues were selected by Northern analysis for comparison purposes. From the open reading frames of the candidate cDNAs we synthesized a dozen peptides which contained a consensus NOD class II MHC binding motif (54, 55). We identified 4 peptides (sequences provided above) from 3 different mouse B cell proteins that were immunogenic in NOD mice (FIG. 4). We did not detect spontaneous proliferative splenic T cell recall responses to these peptides at any stage of NOD mouse development, defining these peptides as NTTAs.
Peptide 2—derived from calbindin D28, a protein which is highly expressed in β cells but which is also expressed at moderate levels in many other tissues.
Peptide 4—from cDNA of unknown function which is expressed at low levels in B cells and appears to be B cell-specific based on northern analysis.
Peptide 6—from another cDNA of unknown function which is expressed at moderate levels and also appears to be B cell-specific in its expression.
Peptide 7—another determinant from the same cDNA as peptide 6.

The remaining peptides tested were peptide 1, a 15mer from calbindin having the sequence EEFMKTWRKYDTDHS;
peptide 3, also a 15mer from calbindin having the sequence LKDLCEKNKQELDIN;
peptide 5, a 15mer from clone 38 in turn derived from a beta cell specific cDNA library (Neophytou, P. et al Diabetes, 45:127, 1996) having the sequence ILKMDHHCPWVNNCV; and
peptide 8 from islet amyloid polypeptide having the sequence GKRNAAGDPNRESLDFL. These peptides did not contain determinants.

In addition, using a set of overlapping GAD peptides, two GAD peptides have been identified which are immunogenic in NOD mice (GADp18 and GADp27; sequences provided under ANTIGENS, above) but which are ignored by their spontaneous autoimmune responses (data not shown). Together with the foregoing NTTAs described above, these neglected GAD determinants provide substances which can be used to demonstrate that self-tolerance is established to some autoantigen determinants, while other determinants from the same protein become involved in the autoimmune cascade. Furthermore, as immunotherapy based on administering neglected GAD determinants is greatly superior to GAD target determinants at late stages of the disease process (see below), these results demonstrate that regulatory tolerance can be elicited even when other tolerizers fail. These conclusions can be used to advantage for the development of human NTTAs as immunotherapeutics for autoimmune disease, and other abnormal inflammatory immune responses.

The foregoing NTTAs may themselves be inappropriate for human immunotherapy because the human MHC is different from mouse MHC. However, using the methods described herein, additional NTTAs can be identified which can be used for humans. The foregoing array of NOD model NTTAs have different expression patterns. Their performance as tolerizers can be used for guidance to select antigen based human immunotherapeutics according to the hierarchy of β cell antigen determinants. The use of peptides (rather than whole antigens) is preferred because the potential variables are limited and their therapeutic efficacy can be more conveniently evaluated.

The foregoing method can be applied for identifying additional NTTAs in any tissue affected by inflammation.

### EXAMPLE 5

### Treatment with NTTAs Prevents the Adoptive Transfer of IDDM

Splenic T cells from NTTA peptide treated mice were co-transferred with T cells from diabetic NOD mice into irradiated young NOD mice. All of the mice which received cells from mice treated with a control immunogenic mouse serum albumin peptide (MSA 560-574) developed IDDM within 5 weeks. In contrast, all of the groups which received T cells from NTTA-treated mice had significantly reduced IDDM incidence, (n=9-12/group) (FIG. 5). Thus, NTTAs are capable of inducing adoptively transferable regulatory responses which can down-regulate pathogenic T cells.

### EXAMPLE 6

### Treatment with NTTAs, But not with βCAA Target Determinants, Inhibits Disease Progression

Groups of six weeks old female NOD mice were treated with either the control MSA (MSA 560-574) peptide, or a peptide containing a βCAA target determinant (GADp35, GADp34, HSP 277 or insulin B-chain), or a NTTA peptide #2, 4, 6 or 7 (in each case 100 μg ip in IFA)). The mice were boosted once 10 days later. All mouse groups were injected concurrently and housed in the same SPF room. NOD mice tested with the control MSA peptide displayed a disease incidence which was similar to that of unmanipulated NOD mouse groups (FIG. 6). An average of 70% of the mice treated with a βCAA target determinant developed IDDM by 40 weeks in age. Two consecutive blood glucose levels of >300 ng/dl was considered disease onset. N=10 for each group. (Other studies which have shown that insulin B-chain and HSP 277 treatment inhibited disease progression started these treatments at an earlier age, or gave multiple boosts.)

In contrast, treatment with NTTAs significantly inhibited disease compared to the control group (p<0.001 using the log rank test). Furthermore, the groups of mice treated with NTTAs were significantly better protected from disease than groups which had been treated with βCAA target determinants (p<0.0006). An average of 40% of the NOD mice treated with different NTTAs developed IDDM by 40 weeks in age. The NTTA which conferred the least protection was derived from the ubiquitously expressed calbindin molecule. Thus, there is variability in therapeutic efficacy, depending on the administered antigen, but all protective effect is within the scope of the invention.

The data from the groups of mice treated with either NTTAs or βCAAs (shown in FIG. 6) are combined in FIG. 7, giving the combined the time to disease for NTTAs vs. βCAAs. In FIG. 7, NTTA is designated by a broken line. (-------), βCAA by a dotted line (.........), and control MSA by a continuous line (—). Data were combined using Kaplan-Meier analysis.

### EXAMPLE 7

### Neglected Determinants, But not Target Determinants, from the Same Protein Inhibit Disease Progression in NOD Mice with Advanced Disease

12 wk old female NOD mice were treated with either a GAD target determinant (GADp35, GADp34, or GADp32), or a NTTA of GAD, i.e. an immunogenic determinant that was nevertheless neglected by the autoimmune response (GADp18 or GADp27) [how much? ip? how often?] or a positive control MSA 560-574 (100 μg in IFA, ip). The mice were re-boosted 10 days later. While treatment with the autoantigens GADp35 or GADp34 confers protection when administered at birth (27), these treatments did not provide significant protection when administered at 12 weeks in age—there were no significant differences between GADp35 or GADp34 treated mice and control MSA-peptide treated or unmanipulated NOD mice (negative control) (FIG. 8). In contrast, treatment with neglected determinants of GAD significantly inhibited disease compared to control groups (p<0.008). Furthermore, the groups of mice treated with neglected GAD determinants were significantly better protected from disease than groups which had been treated with GAD treated determinants (p<0.01). The averaged incidence of combined groups of mice treated with NTTAs vs. βCAA peptide is shown in FIG. 9. The treated groups were combined using Kaplan-Meier analysis.

### EXAMPLE 8

### Pro-Inflammatory Responses Primed by Autoantigen

NOD mice neonatally treated with (100 μg i.p. in IFA) GAD, insulin B-chain or HSP 277, displayed both Th2 and Th1 responses (assessed by ELISPOT) to the injected autoantigen at 4 weeks in age (FIG. 10). The data are represented as the mean number of spot forming colonies per million splenic T cells above background. The variation within, each group was <15%. Experimental and control mice were tested simultaneously (in triplicate) in two separate experiments (n=5/group).

Normally, NOD mice do not develop detectable Th1 responses to these antigens until several weeks later. The results indicate that these autoantigens not only induced Th2 responses and the associated Th2 spreading to other beta cell autoantigens but it also primed accelerated Th1 responses to the injected antigen since T cells in the NOD model become partially primed to βCAAs shortly after birth. While such T cells would not normally be activated until later in the disease process when a more pro-inflammatory environment is established, the greatly increased presentation of the injected antigen on antigen presenting cells (APC) may have driven T cells which had been partially activated toward the Th1 phenotype, to become fully activated and expand to levels of detectability. Thus, the administration of autoantigens can prime pro-inflammatory response to the injected antigen. It is anticipated that NTTAs will induce polarized Th2 responses, avoiding pro-inflammatory responses.

### EXAMPLE 9

### Generation of NTTA and βCAA-Reactive Clones

NOD mice were immunized with a NTTA peptide or GADp35 in incomplete Freund's adjuvant (IFA). Two weeks later lymph node and splenic T cells were cultured with antigen, IL-2 and IL-4 together with irradiated NOD splenic cells and T cell clones were generated by limiting dilution. A NTTA 7 and a GADp35-reactive clone were characterized by ELISPOT and analysis of antigen-stimulated culture supernatants. Both clones secreted IL-4, and no IFN-γ in response to the corresponding antigen. Thus, these T cell clones appear to be Th2-type.

### EXAMPLE 10

### Cloning and Expression of the Whole Antigens Containing NTTA Determinants

Whole antigens are necessary to determine whether the NTTA peptides constitute dominant or cryptic determinants after the natural processing of whole antigens. The whole antigens which have been determined to contain NTTA (in the case of calbindin D28 and GAD) or the putative open reading frames encoding sequences including the sequences of peptides 4, 6 and 7) have been PCR-cloned, recombinantly expressed and their encoded proteins purified by metal affinity chromatography.

### EXAMPLE 11

### Characterization of Single Antigen-Specific T Cells from Pancreatic Islet Infiltrates

Monocytes were isolated from islets of 10-week old female NOD mice. 1000, 100 or 10 monocytes were mixed with 1 million irradiated splenic T cells and tested for IFNγ and IL-5 responses to B cell autoantigens, as well as control antigens, by ELISPOT. The results are shown in Table I as number of T cells. These data demonstrate that low frequency antigen-specific T cells within islet infiltrates can be characterized and used to assess participation of an antigen in autoimmunity.

TABLE 1

| | |
|---|---|
| IFNγ | IL-5 |

| | 1,000 | 100 | 10 | 1,000 | 100 | 10 |
|---|---|---|---|---|---|---|
| GAD | 123 | 15 | — | — | — | — |
| HSP | 5 | — | — | — | — | — |
| Insulin B-chain | 50 | 6 | — | — | — | — |
| β-galactosidase | — | — | — | — | — | — |
| anti-CD3 | 650 | 71 | 6 | 561 | 58 | 5 |

Example of Human NTTA Identification and Treatment. For individuals that are positive for anti-islet cell autoantibodies, or both anti-islet cell antibodies and anti-insulin autoantibodies, or for individuals (e.g. in the “honeymoon period” of IDDM) who still have ongoing autoimmune responses, the following procedure can be followed:

Whole IAPP (or calbindin) can be selected and tested as the putative NTTA. Peripheral lymphocytes from patients can be exposed to antigen, and their proliferation can be measured (by proliferation assay) or their cytokine profile can be assessed (e.g. by ELISPOT) or antibody secretion can be quantified (e.g. by ELISA or other immunoassay).

If, contrary to expectation, IAPP is not NTTA, it can be probed for NTTA determinants using peptides comprising segments of the sequence of IAPP.

The method of Example 4 can be followed and sections of IAPP can be probed for fitting the HLA binding motif, and peptides including these sections can be constructed and tested in the same manner. See, e.g., Grey, HM et al, Cancer Surv., 1995, 22:37; Rotzschke, O et al., Curr. Opin. Immunol. 1994 6: 45. A thus selected NTTA is then administered to patients as follows: subcutaneously (or subdermally) 1 mg whole IAPP (or a peptide fragment thereof determined to be NTTA) in alum adjuvant with a booster of the same dose administered two weeks thereafter.

Benefits can be assessed by repeated assaying for stabilization of insulin and/or blood glucose and/or amylin levels and/or advanced glycosylation end products. A decrease in the level of inflammatory cytokines from peripheral lymphocytes 2-4 weeks after the second dose would also signal a benefit. Similarly, in the case of patients that do not yet have hypoinsulinemia or hyperglycemia but have anti-islet antibodies and anti-insulin antibodies, a decrease in circulating auto antibody levels, or a failure to develop overt diabetes within at least a six-month period, (and preferably a one (1) year period) is considered a success.

Alternative embodiments of this invention will be recognized by those skilled in the art and are intended to be included within the scope of the claims.

### REFERENCES

1. Bowman, M. A., E. H. Leiter, and M. A. Atkinson. 1994. Prevention of diabetes in the NOD mouse: implications for therapeutic intervention in human disease. Immunol Today 15:115-20.
2. Tian, J., and D. L. Kaufman, 1998. Attenuation of inducible Th2 immunity with autoimmune disease progression. J. Immunol. 161:5399-5403.
3. Tian, J., M. Clare-Salzler, A. Herschenfield, B. Middleton, D. Newman, R. Mueller, S. Arita, C. Evans, M. A. Atkinson, Y. Mullen, N. Sarvetnick, A. J. Tobin, P. V. Behmann, and D. L. Kaufman, 1996. Modulating autoimmune responses to GAD inhibits disease progression and prolongs islet graft survival in diabetes-prone mice. Nat. Med 2:1348-53.
4. Oehen, S. U., P. S. Ohashi, K. Burki, H. Hengartner, R. M. Zinkernagel, and P. Aichele. 1994. Escape of thymocytes and mature T cells from clonal deletion due to limiting tolerogen expression levels. Cell Immunol. 158:432-52.
5. Zal. T., A. Volkmann, and B. Stockinger. 1994. Mechanisms of tolerance induction in major histocompatibility complex class II-restricted T cells specific for a blood-borne self-antigen. J. Exp. Med. 180:2089-99.
6. Von Herrath, M. G., J. Dockter, and M. B. Oldstone. 1994. How virus induces a rapid or slow onset insulin-dependent diabetes mellitus in a transgenic model. Immunity 1:231-42.
7. Akkaraju, S., W. Y. Ho, D. Leong, K. Canaan, M. M. Davis, and C. C. Goodnow. 1997. A range of CD4 T cell tolerance: partial inactivation to organic-specific antigen allows nondestructive thyroiditis or insulitis. Immunity 7:255-71.
8. Cibotti, R., J. M. Kanellopoulos, J. P. Cabaniols, O. Halle-Panenko, K. Kosmatopoulos, E. Sercarz, and P. Kourilsky. 1992. Tolerance to a self-protein involves its immunodominant but does not involve its subdominant determinants. Proc. Natl. Acad. Sci. U.S.A. 89:416-20.
9. Mosmann, T. R., and S. Sad. 1996. The expanding universe of T-cell subsets: Th1, Th2 and more. Immunol. Today 17:138-46.
10. Abbas, A. K., K. M. Murphy, and A. Sher. 1996. Functional diversity of helper T lymphocytes. Nature 383:787-93.
11. Weiner, H. L. 1997. Oral tolerance: Immune mechanisms and treatment of autoimmune diseases. Immunology Today 18:335-43.
12. Groux, H., A O'Garra, M. Bigler, M. Rouleau, S. Antonenko, J. E. de Vries, and M. G. Roncarolo. 1997. A CD4+ T-cell subset inhibits antigen-specific T-cell responses and prevents colitis. Nature 389:737-42.
13. Liblau, R. S., S. M. Singer, and H. O. McDevitt. 1995. Th1 and Th2 CD4+ T cells in the pathogenesis of organ-specific autoimmune diseases. Immunol Today 16:34-8.
14. Powrie, F., and R. L. Coffman. 1993. Cytokine regulation of T-cell function: potential for therapeutic intervention. Immunol. Today 14:270-4.
15. Rocken, M., M. Racke, and E. M. Shevach. 1996. IL-4-induced immune deviation as antigen specific therapy for inflammatory autoimmune disease. Immunol. Today 17:225-31.
16. Steinman, L. 1996. A few autoreactive cells in an autoimmune filtrate control a vast population of nonspecific cells: a tale of smart bombs and the infantry. Proc. Natl. Acad. Sci. U.S.A. 93:2253-6.
17. Tian, J., A. P. Olcott, L. R. Hanssen, D. Zekzer, B. Middleton, and D. L. Kaufman. 1998 Infectious Th1 and Th2 immunity in diabetes prone mice. Immunological Reviews 164:119-127.
18. Mueller, R., T. Krahl, and N. Sarvetnick. 1996. Pancreatic expression of interleukin-4 abrogates insulitis and autoimmune diabetes in nonobese diabetic (NOD) mice. J. Exp. Med. 184:1093-9.
19. Elias, D., and I. R. Cohen. 1994. Peptide therapy for diabetes in NOD mice. Lancet 343:704-6.
20. Elliot, J. F., H. Y. Win, S. Bhatti, D. K. Smith, R. K. Singh, T. Dillon, J. Lauzon, and B. Singh. 1994. Immunization with the larger isoform of mouse glutamic acid decarboxylase (GAD67) prevents autoimmune diabetes in NOD mice. Diabetes 43:1494-9.
21. Daniel, D., and D. R. Wegmann. 1996. Protection of nonobese diabetic mice from diabetes by intranasal or subcutaneous administration of insulin peptide B-(9-23). Proc. Natl. Acad. Sci. U.S.A. 93:956-60.
22. Muir, A., A Pedk, M. Clare-Salzler, Y. H. Song, J. Cornelius, R. Luchetta, J. Kriescher, and No. Maclaren. 1995. Insulin immunization of nonobese diabetic mice induces a protective insulitis characterized by diminished intraislet interferon-gamma transcription. J. Clin. Invest. 0.95:628-34.
23. Chao, C. C., and H. O. McDevitt. 1997. Identification of immunogenic ipitopes of GAD 65 presented by Ag7 in non-obese diabetic mice. Immunogenetics 46:29-34,
24. Ashton-Rickardt, P. G., and S. Tonegawa. 1994. A differential-avidity model for T-cell selection. Immunol. Today 15:362-6.
25. Sercarz, E. E., P. V. Lehmann, A. Ametani, G. Benichou, A. Miller, and K. Moudgil. 1993. Dominance and crypticity of T cell antigenic determinants. Annu. Rev. Immunol. 11:729-66.
26. Lehmann, P. V., O. S. Targoni, and T. G. Forsthuber. 1998. Shifting T-cell activation thresholds in autoimmunity and determinant spreading. Immunol. Rev. 164:53-61.
27. Tian, J., P. V. Lehmann, and D. L. Kaufman. 1997. Determinant spreading of t helper 2 (RH2) responses to pancreatic islet autoantigens. J. Exp. Med. 186:2039-2044.
28. Kelso, A. 1995. Th1 and Th2 subsets: paradigms lost? Immunol. Today 16:374-9.
29. Allen, J. E., and R. M. Maizels. 1997. Th1-Th2: Reliable Paradigm or Dangerous Dogma? Immunology Today 18:387-92.
30. Harrison, L. C., M. Dempsey-Collier, D. R. Kramer, and K. Takahashi. 1996. Aerosol insulin induces regulatory CD8 gamma delta t cells that prevent murine insulin-dependent diabetes. J. Exp. Med. 184:2167-74.
31. Cai, A., A. Brunmark, M. R. Jackson, D. Loh, P. A. Peterson, and J. Sprent. 1996. Transfected Drosophila cells as a probe for defining the minimal requirements for stimulating unprinted CD8+ cells. Proc. Natl. Acad. Sci. U.S.A. 93:14736-42.
32. Pihlgren M., P. M. Dubois, m. Tomkowiak, T. Sjogren, and J. Marvel. 1996. Resting memory CD8+ T cells are hyperreactive to antigenic challenge in vitro. J. Exp. Med. 184:2141-51.
33. Dubey, C., M. Croft, and S. L. Swain. 1995. Costimulatory requirements of naive CD4+ T cells. ICAM-1 or B7-1 can costimulate naive CD4 T cell activation but both are required for optimum response. J. Immunol. 155:45-57.
34. Dubey, C., M. Croft, and S. L. Swain. 1995. Naiv and effector CD4 T cells differ in their requirements for T cell receptor versus costimulatory signals. J. Immunol. 157:3280-9.
35. Bachmann, M. F., K. McKall-Faienza, R. Schmits, D. Bouchard, J. Beach, D. E. Speiser, T. W. Mak, and P. S. Ohashi. 1997. Distinct roles for LFA-1 and CD28 during activation of naive T cells: adhesion versus costimulation. Immunity 7:549-57.
36. Kaufman, D. L., M. Clare-Salzler, J. Tian, T. Forsthuber, G. S. Ting, P. Robinson, M. A. Atkinson, E. E. Sercarz, A. J. Tobin, and P. V. Lehmann. 1993. Spontaneous loss of T-cell tolerance to glutamic acid decarboxylase in murine insulin-dependent diabetes. Nature 366:69-72.
37. Atkinson, M. A., M. A. Bowman, L. Campbell. B. L. Darrow, D. L. Kaufman, and N. K. Maclaren. 1994. Cellular immunity to a determinant common to glutamate decarboxylase and coxsackie virus in insulin-dependent diabetes [see comments]. J. Clin. Invest 94:2125-9.
38. Tian, J. M. A. Atkinson, M. Clare-Salzer, A. Herschenfield, T. Forsthuber, P. V. Lehmann, and D. L. Kaufman. 1996. Nasal administration of glutamate decarboxylase (GAD65) peptides induces Th2 responses and prevents murine insulin-dependent diabetes. J. Exp. Med. 183:1561-7.
39. Tian, J., and D. L. Kaufman. 1998. Insulin selectively primes Th2 responses and induces regulatory tolerance to insulin in pre-diabetic mice. Diabetologia 41:237-240.
40. Targoni, O. S., and P. V. Lehmann. (Submitted).
41. Kaufman, D. L., and A. J. Tobin. 1993. Glutamate decarboxylases and autoimmunity in insulin-dependent diabetes. Trends Pharmacol. Sci. 14:107-9.
42. Mueller, R., L. M. Bradley, T. Krahl, and N. Sarvetnick. 1997. Mechanism underling counterregulation of autoimmune diabetes by IL-4. Immunity 7:411-8.
43. Zekzer, D., F. S. Wong, L. Wen, M. Altieri, T. Gurlo, H. von Grafenstein, and R. S. Sherwin. 1997. Inhibition of diabetes by an insulin-reactive CD4 T-cell done in the nonobese diabetic mouse. Diabetes 46:1124-32.
44. Zekzer, D., F. S. Wong, O. Ayalon, 1. Millet, M. Alteiri, S. Shintani, M. Solimena and R. Sherwin 1998. GAD-reactive CD4⁺ Th1 cells induce diabetes in NOD/SCID mice. J. Clin. Invest. 101:68-73.
45. Pakala, S. V., M. O. Kurrer, and J. D. Katz. 1997. Thelper 2 (Th2) T cells induce acute pancreatitis and diabetes in immune-compromised nonobese diabetic (NOD) mice. Journal of Experimental Medicine 186:299-306.
46. Lafaille, J. J., F. Van de Keere, A. L. Hsu, J. L. Baron, W. Haas, C. S. Raine, and S. Tonegawa 1997. Myelin basic protein-specific Thelper 2 (Th2) cells cause experimental autoimmune encephalomyelitis in immodeficient hosts rather than protect them from the disease. Journal of Experimental Medicine 186:307-12.
47. Genain, C. P., K. Abel, N. Belmar, F. Villinger, D. P. Rosenberg, C. Linington, C. S. Raine, and S. L. Hauser. 1996. Late complications of immune deviation therapy in a nonhuman primate. Science 274:2054-7.
48. Blanas, E., F. R. Carbone, J. Allison, J. F. Miller, and W. R. Heath. 1996. Induction of autoimmune diabetes by oral administration of autoantigen. Science 274: 1707-9.
49. Kat, J. D., C. Benoist, and D. Mathis. 1995. T helper cell subsets in insulin-dependent diabetes. Science 268:1185-8.
50. Elias, D., T. Reshef, O. S. Birk, R. van der Zee, M. D. Walker, and I. R. Cohen. 1991. Vaccination against autoimmune mouse diabetes with a T-cell epitope of human 65-kDa heat shock protein. Proc. Natl. Acad. Sci. U.S.A. 88:3088-91.
51. Haskins, K., and D. Wegmann. 1996. Diabetogenic T-cell clones. Diabetes 45:1299-305.
52. Harrison, L. C., M. C. Honeyman, J. J. DeAizpurua, R. S. Schmidli, P. G. Colman, B. D. Tait, and D. S. Cram. 1993. Inverse relation between humoral and cellular immunity to glutamic acid decarboxylase in subjects at risk of insulin-dependent diabetes [see comments]. Lancet 341:1365-9.
53. Thai, A. C., and G. S. Eisenbarth. 1993. Natural history of IDDM. Diabetes Reviews 1:1-14.
   54. Reicht, E. P., H. von Grafenstein, A Barlow, K. E. Swenson, K. Williams, and C. A. Janeway, Jr. 1994. Self peptides isolated from MHC glycoproteins of non-obese diabetic mice. J. Immunol. 152:2279-88.
55. Harrison, L. C., M. C. Honeyman, S. Trembleau, S. Gregori, F. Gallazzi, P. Augstein V. Brusic, J. Hammer, and L. Adorini. 1997. A peptide-binding motif for I-A(g7), the class II major histocompatibility complex (MHC) molecule of NOD and Biozzi AB/H mice. J. Ex. Med. 185:1013-21.
56. Sharma, S. D., Nag, B., X. M. et al. (1991) Proc. Natl. Acad Sci U.S.A. 88, 11465-11469.
57. Critchfield, J. M., Rackie, M. K., Zuniga-Pflucker, J. C. et al. (1994) Science 263, 1139-1143.

1-19. (canceled)

20. A method for selecting a peptide for treating an autoimmune disease affecting a tissue of a host, comprising:
(a) selecting an antigen expressed by the tissue;
(b) identifying a motif in the antigen recognized by a MHC of the host to be treated;
(c) synthesizing a peptide incorporating the motif, and
(d) determining if the peptide fails to stimulate a T-cell recall response from a host with the disease.

21. The method of claim 20, wherein said autoimmune disease affecting the tissue of the host is at a late stage of autoimmunity.

22. The method of claim 20, wherein said antigen is an autoantigen.

23. The method of claim 22, wherein said autoantigen is selected from the group consisting of SEQ ID NOS: 1-4 and 17-27.

24. The method of claim 20, wherein said antigen is selected from the group consisting of SEQ ID NOS: 5-16.

25. The method of claim 20, wherein said autoimmune disease is selected from the group consisting of insulin-dependent diabetes mellitus, multiple sclerosis, rheumatoid arthritis, uveoretinitis, and autoimmune thyroiditis.

26. A method for selecting a peptide for treating an autoimmune disease affecting a tissue of a host, comprising:
(a) selecting an antigen expressed by the tissue;
(b) determining that a determinant of the antigen fails to stimulate lymphocytes from a host with the disease; and
(c) determining if a host mounts a T-cell response when the host is immunized with a peptide consisting essentially of the determinant.

27. The method of claim 26, wherein said autoimmune disease affecting the tissue of the host is at a late stage of autoimmunity.

28. The method of claim 26, wherein said antigen is an autoantigen.

29. The method of claim 28, wherein said autoantigen is selected from the group consisting of SEQ ID NOS: 1-4 and 17-27.

30. The method of claim 26, wherein said antigen is selected from the group consisting of SEQ ID NOS: 5-16.

31. The method of claim 26, wherein said autoimmune disease is selected from the group consisting of insulin-dependent diabetes mellitus, multiple sclerosis, rheumatoid arthritis, uveoretinitis, and autoimmune thyroiditis.


SEQUENCE LISTING (33 sequences)

SEQ ID NO: 1 - protein, 604 aa, homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDLGKRNAVEVLKREPLNYLPL

SEQ ID NO: 2 - protein, 594 aa, homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 3 - protein, 20 aa, homo sapiens
PEVKEKGMAAVPRLIAFTSE

SEQ ID NO: 4 - protein, 20 aa, homo sapiens
PLQCSALLVREEGLMQSCNQ

SEQ ID NO: 5 - protein, 76 aa, bos taurus
MKSPEELKGIFEKYAAKEGDPNQLSKEELKLLLQTEFPSLLKGGSTLDELFEELDKNGDGEVSFEEFQVLVKKISQ

SEQ ID NO: 6 - protein, 90 aa, oryctolagus cuniculus
MASPLDQAIGLLIGIFHKYSGKEGDKHTLSKKELKELIQKELTIGSKLQDAEIVKLMDDLDRNKDQEVNFQEYITFLGALAMIYNEALKG

SEQ ID NO: 7 - protein, 271 aa, homo sapiens
MAGPQQQPPYLHLAELTASQFLEIWKHFDADGNGYIEGKELENFFQELEKARKGSGMMSKSDNFGEKMKEFMQKYDKNSDGKIEMAELAQILPTEENFLLCFRQHVGSSAEFMEAWRKYDTDRSGYIEANELKGFLSDLLKKANRPYDEPKLQEYTQTILRMFDLNGDGKLGLSEMSRLLPVQENFLLKFQGMKLTSEEFNAIFTFYDKDRSGYIDEHELDALLKDLYEKNKKEINIQQLTNYRKSVMSLAEAGKLYRKDLEIVLCSEPPM

SEQ ID NO: 8 - protein, 261 aa, homo sapiens
MAESHLQSSLITASQFFEIWLHFDADGSGYLEGKELQNLIQELQQARKKAGLELSPEMKTFVDQYGQRDDGKIGIVELAHVLPTEENFLLLFRCQQLKSCEEFMKTWRKYDTDHSGFIETEELKNFLKDLLEKANKTVDDTKLAEYTDLMLKLFDSNNDGKLELTEMARLLPVQENFLLKFQGIKMCGKEFNKAFELYDQDGNGYIDENELDALLKDLCEKNKQDLDINNITTYKKNIMALSDGGKLYRTDLALILCAGDN

SEQ ID NO: 9 - protein, 261 aa, homo sapiens; identical to SEQ ID NO: 8

SEQ ID NO: 10 - protein, 20 aa, homo sapiens
ELKNFLKDLLEKANKTVDDT

SEQ ID NO: 11 - protein, 16 aa, homo sapiens
LIKPDRCHHCSVCDKC

SEQ ID NO: 12 - protein, 89 aa, homo sapiens
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 13 - protein, 70 aa, homo sapiens
MGILKLQVFLIVLSVALNHLKATPIESHQVEKRKCNTATCATQRLANFLVHSSNNFGAILSSTNVGSNTY

SEQ ID NO: 14 - protein, 15 aa, homo sapiens
NHLRATPVRSGSNPQ

SEQ ID NO: 15 - protein, 15 aa, homo sapiens
TQRLANFLVRSSNNL

SEQ ID NO: 16 - protein, 97 aa, homo sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 17 - protein, 359 aa, homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 18 - protein, 354 aa, homo sapiens
MKSLLLLVLISFCWADHHSDNYTVDHDRVIHIQAENGPRLLVEAEQAKVFSRRGGNVTLPCKFYRDPTAFGSGTHKIRIKWTKLTSDYLKEVDVFVSMGYHKKTYGGYHGRVFLKGGSDNDASLVITDLTLEDYGRYKCEVIEGLEDDTAVVALDLQGVVFPYFPRLGRYNLNFHEAQQACLDQDAVIASFDQLYDAWRSGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKLLGYDRCDAGWLADGSVRYPISRPRRRCSPSEAAVRFVGFPDKKHKLYGVYCFRAYN

SEQ ID NO: 19 - protein, 354 aa, homo sapiens
MKSLLLLVLISICWADHLSDNYTLDHDRAIHIQAENGPHLLVEAEQAKVFSHRGGNVTLPCKFYRDPTAFGSGIHKIRIKWTKLTSDYLKEVDVFVSMGYHKKTYGGYQGRVFLKGGSDSDASLVITDLTLEDYGRYKCEVIEGLEDDTVVVALDLQGVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

SEQ ID NO: 20 - protein, 355 aa, gallus gallus
MTSLLFLVLISVCWAEPHPDNSSLEHERIIHIQEENGPRLLVVAEQAKIFSQRGGNVTLPCKFYHEHTSTAGSGTHKIRVKWTKLTSDYLKEVDVFVAMGHHRKSYGKYQGRVFLRESSENDASLIITNIMLEDYGRYKCEVIEGLEDDTAVVALNLEGVVFPYSPRLGRYNLNFHEAQQACLDQDSIIASFDQLYEAWRSGLDWCNAGWLSDGSVQYPITKPREPCGGKNTVPGVRNYGFWDKERSRYDVFCFTSNFNGRFYYLIHPTKLTYDEAVQACLKDGAQIAKVGQIFAAWKLLGYDRCDAGWLADGSVRYPISRPRKRCSPNEAAVRFVGFPDKKHKLYGVYCFRAYN

SEQ ID NO: 21 - protein, 339 aa, rattus norvegicus
DHLSDSYTPDQDRVIHIQAENGPRLLVEAEQAKVFSHRGGNVTLPCKFYRDPTAFGSGIHKIRIKWTKLTSDYLREVDVFVSMGYHKKTYGGYQGRVFLKGGSDNDASLIITDLTLEDYGRYKCEVIEGLEDDTAVVALELQGVVFPYFPRLGRYNLNFHEARQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKLLGYDRCDAGWLADGSVRYPISRPWRRCSPTEAAVRFVGFPDKKHKLYGVYCFRAYN

SEQ ID NO: 22 - protein, 354 aa, homo sapiens; identical to SEQ ID NO: 19

SEQ ID NO: 23 - protein, 563 aa, homo sapiens
MDFGSLETVVANSAFIAARGSFDGSSSQPSRDKKYLAKLKLPPLSKCESLRDSLSLEFESVCLEQPIGKKLFQQFLQSAEKHLPALELWKDIEDYDTADNDLQPQKAQTILAQYLDPQAKLFCSFLDEGIVAKFKEGPVEIQDGLFQPLLQATLAHLGQAPFQEYLGSLYFLRFLQWKWLEAQPMGEDWFLDFRVLGKGGFGEVSACQMKATGKLYACKKLNKKRLKKRKGYQGAMVEKKILMKVHSRFIVSLAYAFETKADLCLVMTIMNGGDIRYHIYNVNEENPGFPEPRALFYTAQIICGLEHLHQRRIVYRDLKPENVLLDNDGNVRISDLGLAVELLDGQSKTKGYAGTPGFMAPELLQGEEYDFSVDYFALGVTLYEMIAARGPFRARGEKVENKELKHRIISEPVKYPDKFSQASKDFCEALLEKDPEKRLGFRDETCDKLRAHPLFKDLNWRQLEAGMLMPPFIPDSKTVYAKDIQDVGAFSTVKGVAFDKTDTEFFQEFATGNCPIPWQEEMIETGIFGELNVWRSDGQMPDDMKGISGGSSSSSKSGMCLVS

SEQ ID NO: 24 - protein, 354 aa, danio rerio
MNGTEGPAFYVPMSNATGVVRSPYEYPQYYLVAPWAYGLLAAYMFFLIITGFPVNFLTLYVTIEHKKLRTPLNYILLNLAIADLFMVFGGFTTTMYTSLHGYFVFGRLGCNLEGFFATLGGEMGLWSLVVLAIERWMVVCKPVSNFRFGENHAIMGVAFTWVMACSCAVPPLVGWSRYIPEGMQCSCGVDYYTRTPGVNNESFVIYMFIVHFFIPLIVIFFCYGRLVCTVKEAAAQQQESETTQRAEREVTRMVIIMVIAFLICWLPYAGVAWYIFTHQGSEFGPVFMTLPAFFAKTSAVYNPCIYICMNKQFRHCMITTLCCGKNPFEEEEGASTTASKTEASSVSSSSVSPA

SEQ ID NO: 25 - protein, 604 aa, mus musculus
LVLSTLLWVPAGPTHLFQPSLVLDMAKILLDNYCFPENLMGMQAAIEQAMKSHEILGISDPQTLAQVLTAGVQSSLSDPRLFISYEPSTLEAPQQAPVLTNLTREELLAQIQRNIRHEVLEGNVGYLRVDDLPGQEVLSELGEFLVSHVWRQLMSTSSLVLDLRHCSGGHFSGIPYVISYLHPGNTVMHVDTVYDRPSNTTTEIWTLPEVLGERYSADKDVVVLTSGHTGGVAEDIAYILKQMRRAIVVGERTEGGALDLQKLRIGQSNFFLTVPVSRSLGPLGGGGQTWEGSGVLPCVGTPAEQALEKALAILTLRRALPGVVLRLQEALQDYYTLVDRVPGLLHHLASMDYSAVVSEEDLVTKLNAGLQAVSEDPRLLVRATGPRDSSSRPETGPNESPAATPEVPTEEDARRALVDSVFQVSVLPGNVGYLRFDRFADAAVLETLGPYVLKQVWEPLQDTEHLIMDLRHNPGGPSSAMPLVLSYFQGPEAGPVRLFTTYDRRTNITQEHFSHRELLGQRYGNQRGVYLLTSHRTATAAEEFAFLMQSLGWATLVGEITAGSLLHTCTVPLLDSPQGGLALTVPVLTFIDNHGEAWLGGGVV

SEQ ID NO: 26 - protein, 105 aa, gallus gallus
  UNSURE (103): Xaa is unknown or other
DMRFNIGGYTNWIPILCSYFFDAGHQVLLDKVYDRPSDSVKEIWTQPQLRGERYGSQKGLIILTSAVTAGAAEEFVFIMKRLGRALIIGEQTSGGSHSPQTYXVD

SEQ ID NO: 27 - protein, 771 aa, homo sapiens
MYLETRRAIFVFWIFLQVQGTKDISINIYHSETKDIDNPPRNETTESTEKMYKMSTMRRIFDLAKHRTKRSAFFPTGVKVCPQESMKQILDSLQAYYRLRVCQEAVWEAYRIFLDRIPDTGEYQDWVSICQQETFCLFDIGKNFSNSQEHLDLLQQRIKQRSFPDRKDEISAEKTLGEPGETIVISTDVANVSLGPFPLTPDDTLLNEILDNTLNDTKMPTTERETEFAVLEEQRVELSVSLVNQKFKAELADSQSPYYQELAGKSQLQMQKIFKKLPGFKKIHVLGFRPKKEKDGSSSTEMQLTAIFKRHSAEAKSPASDLLSFDSNKIESEEVYHGTMEEDKQPEIYLTATDLKRLISKALEEEQSLDVGTIQFTDEIAGSLPAFGPDTQSELPTSFAVITEDATLSPELPPVEPQLETVDGAEHGLPDTSWSPPAMASTSLSEAPPFFMASSIFSLTDQGTTDTMATDQTMLVPGLTIPTSDYSAISQLALGISHPPASSDDSRSSAGGEDMVRHLDEMDLSDTPAPSEVPELSEYVSVPDHFLEDTTPVSALQYITTSSMTIAPKGRELVVFFSLRVANMAFSNDLFNKSSLEYRALEQQFTQLLVPYLRSNLTGFKQLEILNFRNGSVIVNSKMKFAKSVPYNLTKAVHGVLEDFRSAAAQQLHLEIDSYSLNIEPADQADPCKFLACGEFAQCVKNERTEEAECRCKPGYDSQGSLDGLEPGLCGLAQRNARSSRERELHAVPDHSENQAYKTSVKSSKINKITR

SEQ ID NO: 28 - protein, 186 aa, homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 29 - protein, 433 aa, homo sapiens
  UNSURE (14): Xaa is unknown or other
MSLCLFILLFLTPXILCICPLQCICTERHRHVDCSGRNLSTLPSGLQENIIHLNLSYNHFTDLHNQLTQYTNLRTLDISNNRLESLPAHLPRSLWNMSAANNNIKLLDKSDTAYQWNLKYLDVSKNMLEKVVLIKNTLRSLEVLNLSSNKLWTVPTNMPSKLHIVDLSNNSLTQILPGTLINLTNLTHLYLHNNKFTFIPDQSFDQLFQLQEITLYNNRWSCDHKQNITYLLKWMMETKAHVIGTPCSTQISSLKEHNMYPTPSGFTSSLFTVSGMQTVDTINSLSVVTQPKVTKIPKQYRTKETTFGATLSKDTTFTSTDKAFVPYPEDTSTETINSHEAAAATLTIHLQDGMVTNTSLTSSTKSSPTPMTLSITSGMPNNFSEMPQQSTTLNLWREETTTNVKTPLPSVANAWKVNASFLLLLNVVVMLAV

SEQ ID NO: 30 - protein, 15 aa, homo sapiens
EEFMKTWRKYDTDHS

SEQ ID NO: 31 - protein, 15 aa, homo sapiens
LKDLCEKNKQELDIN

SEQ ID NO: 32 - protein, 15 aa, mus musculus
ILKMDHHCPWVNNCV

SEQ ID NO: 33 - protein, 17 aa, mus musculus
GKRNAAGDPNRESLDFL